(19) United States
(12) Patent Application Publication
(10) Pub. No.: US 2008/0145698 A1
(43) Pub. Date: Jun. 19, 2008

(54) Compounds For Organic Electronic Devices

(75) Inventors: Holger Heil, Darmstadt (DE); Philipp Stossel, Frankfurt (DE); Horst Vestweber, Gilserberg (DE)

Correspondence Address: CONNOLLY BOVE LODGE & HUTZ, LLP, P O BOX 2207, WILMINGTON, DE, 19899, US

(73) Assignee: Merck Patent GmbH, Darmstadt (DE)

(21) Appl. No.: 11/630,223

(22) PCT Filed: Jun. 22, 2005
(86) PCT No.: PCT/EP05/06728

§ 371 Date: Dec. 20, 2006

(30) Foreign Application Priority Data
Jun. 26, 2004 (DE) 102004031000.9
Jan. 31, 2005 (EP) 05001891.0

(51) Int. Cl. C07D 403/14; C07C 211/54; C07F 9/28; C09K 11/06; G03G 5/147; C07D 401/14; C07D 409/14

(52) U.S. Cl. 428/690; 564/427; 568/17; 546/268.1; 549/59; 549/58; 549/43; 544/357; 546/140; 548/131

(57) ABSTRACT

The invention relates to the improvement of organic electroluminescent devices, especially blue-emitting devices, by using compounds of formula (1)

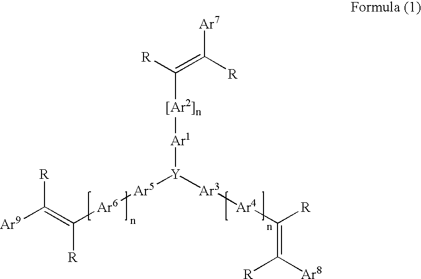

as dopants in the emitting layer.

The present invention describes novel compounds and the use thereof in organic electroluminescent devices.

The use of semiconducting organic compounds which are capable of the emission of light in the visible spectral region in organic electroluminescent devices (OLEDS) is just at the beginning of the market introduction. The general structure of devices of this type is described, for example, in U.S. Pat. No. 4,539,507, U.S. Pat. No. 5,151,629, EP 0676461 and WO 98/27136. For devices containing simple OLEDs, the market introduction has already taken place, as confirmed by the car radios from Pioneer, the mobile telephones from Pioneer and SNMD or a digital camera from Kodak with an “organic” display. Further products of this type are just about to be introduced.

However, these devices still exhibit considerable problems which require urgent improvement:

1. The efficiency is still too low, in particular in the case of fluorescent OLEDs, and must be improved.
2. The operating lifetime is still short, in particular in the case of blue emission, meaning that it has hitherto only been possible to achieve simple applications commercially.
3. The operating voltage is fairly high, in particular in the case of fluorescent OLEDs, and should therefore be reduced further in order to improve the power efficiency. This is of major importance, in particular, for mobile applications.
4. Many blue-emitting emitters which comprise both aromatic amines and also double-bond systems are thermally unstable and decompose on sublimation or on vapour deposition. The use of these systems is thus only possible with major losses and high technical complexity, or not at all.

As closest prior art, mention may be made of the use of certain arylvinylamines by Idemitsu (for example WO 04/013073, WO 04/016575, WO 04/018587), in which very good lifetimes with dark-blue emission are quoted. However, these results, as is evident, are highly dependent on the host material used, meaning that the quoted lifetimes cannot be compared as absolute values, but instead only on use in an optimised system. Furthermore, these compounds are thermally unstable and cannot be evaporated without decomposition, which requires high technical complexity for the vapour deposition and thus represents a significant industrial disadvantage. A further disadvantage is the emission colour of these compounds. While Idemitsu quotes dark-blue emission (CIE y coordinates in the range 0.15-0.18), it has not been possible to reproduce these colour coordinates in simple devices in accordance with the prior art. On the contrary, green-blue emission is obtained here. It is not apparent how blue emission can actually be produced using these compounds.

Thus, there continues to be a demand for blue-emitting compounds which result in good efficiencies and at the same time in long lifetimes in organic electroluminescent devices and which can be processed industrially without problems. Surprisingly, it has now been found that organic electroluminescent devices which comprise certain compounds—mentioned below—as blue-emitting dopants in a host material have significant improvements over the prior art. Using these materials, it is possible to obtain longer lifetimes at the same time as higher efficiency. In addition, these compounds, in contrast to materials in accordance with the prior art, can be sublimed and vapour-deposited without significant decomposition and are therefore significantly easier to handle than materials in accordance with the prior art. The present invention therefore relates to these compounds and to the use thereof in OLEDs.

The invention relates to compounds of the formula (1)

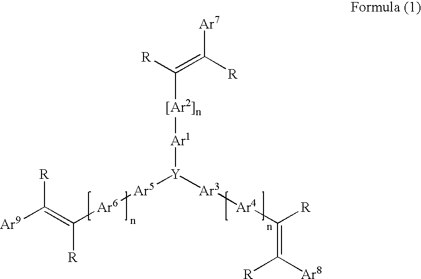

where the following applies to the symbols and indices used:
Y is on each occurrence nitrogen, phosphorus, arsenic, antimony, P═O, As═O or Sb═O;
Ar¹, Ar², Ar³, Ar⁴, Ar⁵, Ar⁶ is on each occurrence, identically or differently, a divalent aryl or heteroaryl group having 2 to 24 C atoms, which may be substituted by one or more radicals R¹;
Ar⁷, Ar⁸, Ar⁹ is on each occurrence, identically or differently, a monovalent aryl or heteroaryl group having 2 to 24 C atoms, which may be substituted by one or more radicals R¹;
R is on each occurrence, identically or differently, H, CN, a straight-chain, branched or cyclic alkyl group having 1 to 40 C atoms, which may be substituted by one or more radicals R², where one or more non-adjacent CH₂ groups may be replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms may be replaced by F, Cl, Br, I, CN or NO₂, or a monovalent aryl or heteroaryl group having 2 to 24 C atoms, which may be substituted by one or more radicals R¹;
R¹ is on each occurrence, identically or differently, H, F, Cl, Br, I, CN, NO₂, a straight-chain, branched or cyclic alkyl, alkoxy or thioalkoxy group having 1 to 40 C atoms, which may be substituted by one or more radicals R², where one or more non-adjacent CH₂ groups may be replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms may be replaced by F, Cl, Br, I, CN or NO₂, or an aryl or heteroaryl group having 2 to 24 C atoms, which may be substituted by one or more radicals R², or an aryloxy or heteroaryloxy group having 2 to 24 aromatic C atoms, which may be substituted by one or more radicals R², or a combination of two, three, four or five of these systems; two or more substituents R¹ here, both on the same ring and also on different rings, may also form a mono- or polycyclic, aliphatic or aromatic ring system with one another;
R² is on each occurrence, identically or differently, H or an aliphatic or aromatic hydrocarbon radical having 1 to 20 C atoms;
n is on each occurrence, identically or differently, 1, 2, 3, 4 or 5.

For the purposes of this invention, an aryl group or heteroaryl group is taken to mean an aromatic group or heteroaromatic group respectively having a common aromatic electron system. For the purposes of this invention, this can be a simple homo- or heterocycle, for example benzene, pyridine, thiophene, etc., or it can be a condensed aromatic ring system in which at least two aromatic or heteroaromatic rings, for example benzene rings, are “fused” to one another, i.e. are condensed onto one another by anellation, i.e. have at least one common edge and thus also a common aromatic system. These aryl or heteroaryl groups may be substituted or unsubstituted; likewise, any substituents present may form further ring systems. Thus, for example, systems such as naphthalene, anthracene, phenanthrene, pyrene, etc., are to be regarded as aryl groups and quinoline, acridine, benzothiophene, carbazole, etc., as heteroaryl groups for the purposes of this invention, while, for example, biphenyl, fluorene, spirobifluorene, etc., do not represent aryl groups since these involve separate aromatic electron systems.

For the purposes of the present invention, a C₁- to C₄₀-alkyl group, in which, in addition, individual H atoms or CH₂ groups may be substituted by the above-mentioned groups, is particularly preferably taken to mean the radicals methyl, ethyl, n-propyl, i-propyl, n-butyl, i-butyl, s-butyl, t-butyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. A C₁- to C₄₀-alkoxy group is particularly preferably taken to mean methoxy, ethoxy, n-propoxy, i-propoxy, n-butoxy, i-butoxy, s-butoxy, t-butoxy or 2-methylbutoxy. A C₂-C₂₄-aryl or -heteroaryl group, which may be monovalent or divalent depending on the use, which may, in addition, in each case be substituted by the above-mentioned radicals R¹ and which may be linked to the aromatic or heteroaromatic ring system via any desired positions, is taken to mean, in particular, groups derived from benzene, naphthalene, anthracene, phenanthrene, pyrene, chrysene, perylene, fluoranthene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

The systems formed by combination of these systems and formation of additional ring systems are preferably biphenylene, terphenylene, fluorene, spirobifluorene, dihydrophenanthrene, dihydropyrene, tetrahydropyrene or cis- or trans-indenofluorene.

Preference is given to compounds of the formula (1) in which the symbol Y stands for nitrogen, phosphorus or P═O, particularly preferably for nitrogen or phosphorus, very particularly preferably for nitrogen.

Preference is furthermore given to compounds of the formula (1) in which the symbols Ar¹, Ar², Ar³, Ar⁴, Ar⁵ and Ar⁶, identically or differently on each occurrence, stand for a divalent aryl or heteroaryl group having 2 to 16 C atoms, which may be substituted by one or two radicals R¹, particularly preferably for a divalent aryl or heteroaryl group having 4 to 14 C atoms, which may be substituted by one or two radicals R¹, very particularly preferably for a divalent aryl or heteroaryl group selected from benzene, naphthalene, anthracene, phenanthrene, pyridine and thiophene, in particular benzene, each of which may be substituted by one or two radicals R¹.

Preference is furthermore given to compounds of the formula (1) in which the symbols Ar⁷, Ar⁸ and Ar⁹, identically or differently on each occurrence, stand for a monovalent aryl or heteroaryl group having 2 to 16 C atoms, which may be substituted by one or more radicals R¹, particularly preferably for a monovalent aryl or heteroaryl group having 4 to 14 C atoms, which may be substituted by one or more radicals R¹, very particularly preferably for a monovalent aryl or heteroaryl group selected from benzene, naphthalene, anthracene, phenanthrene, pyridine and thiophene, in particular benzene, each of which may be substituted by a radical R¹, in particular by an aromatic radical R¹.

Preference is furthermore given to compounds of the formula (1) in which the symbol R, identically or differently on each occurrence, stands for H, CN, a straight-chain or branched alkyl group having 1 to 4 C atoms, in which one or more non-adjacent CH₂ groups may be replaced by —R²C═CR²—, —C≡C—, —O— or —S— and in which one or more H atoms may be replaced by F, or a monovalent aryl or heteroaryl group having 2 to 16 C atoms, which may be substituted by one or more radicals R¹, particularly preferably for H, CN, methyl or a monovalent aryl or heteroaryl group having 4 to 6 C atoms, which may be substituted by one or more radicals R¹, very particularly preferably for H.

Preference is furthermore given to compounds of the formula (1) in which the symbol R¹, identically or differently on each occurrence, stands for H, F, CN, a straight-chain, branched or cyclic alkyl or alkoxy group having 1 to 10 C atoms, which may be substituted by one or more radicals R², where one or more non-adjacent CH₂ groups may be replaced by —R²C═CR²—, —C═C—, Si(R²)₂, C═O, —O— or —S— and where one or more H atoms may be replaced by F, or an aryl or heteroaryl group having 2 to 16 C atoms, which may be substituted by one or more radicals R², or an aryloxy or heteroaryloxy group having 2 to 16 aromatic C atoms, which may be substituted by one or more radicals R², or a combination of two, three or four of these systems; two or more substituents R¹ here, both on the same ring and also on different rings, may also form a mono- or polycyclic, aliphatic or aromatic ring system with one another.

Preference is furthermore given to compounds in which the index n, identically or differently on each occurrence, stands for 1, 2 or 3, preferably for 1 or 2, very particularly preferably for 1.

The divalent aryl or heteroaryl groups Ar¹ and Ar² or Ar³ and Ar⁴ or Ar⁵ and Ar⁶ are preferably linked in such a way that an even number of aromatic ring atoms is located between the two linking points, in particular a number of ring atoms which can be divided by four. Thus, for phenylene systems, for example, ortho- and para-linking are preferred, in particular para-linking.

Preference is given to compounds of the formula (1) in which Ar¹, Ar³ and Ar⁵ stand for the same aryl or heteroaryl group. Preference is furthermore given to compounds of the formula (1) in which Ar², Ar⁴ and Ar⁶ stand for the same aryl or heteroaryl group. Preference is furthermore given to compounds of the formula (1) in which Ar⁷, Ar⁸ and Ar⁹ stand for the same aryl or heteroaryl group.

Particular preference is given to compounds of the formula (1) which have a symmetrical structure and a three-fold axis of rotation (C3), which relates not only to the aromatic groups Ar¹ to Ar⁹, but also to the radicals R, R¹ and R². This preference is due to the easier synthetic accessibility of the compounds. However, the asymmetrical compounds are also accessible in more steps.

If the compounds are able to exhibit atropisomerism about one or more bonds, the invention in each case also relates to the isolated or enriched atropisomers. This relates both to enantiomers and also to diastereomers. The choice of suitable atropisomers enables, for example, the solubility of the compound to be influenced.

Examples of preferred compounds of the formula (1) are Examples 1 to 42 depicted below.

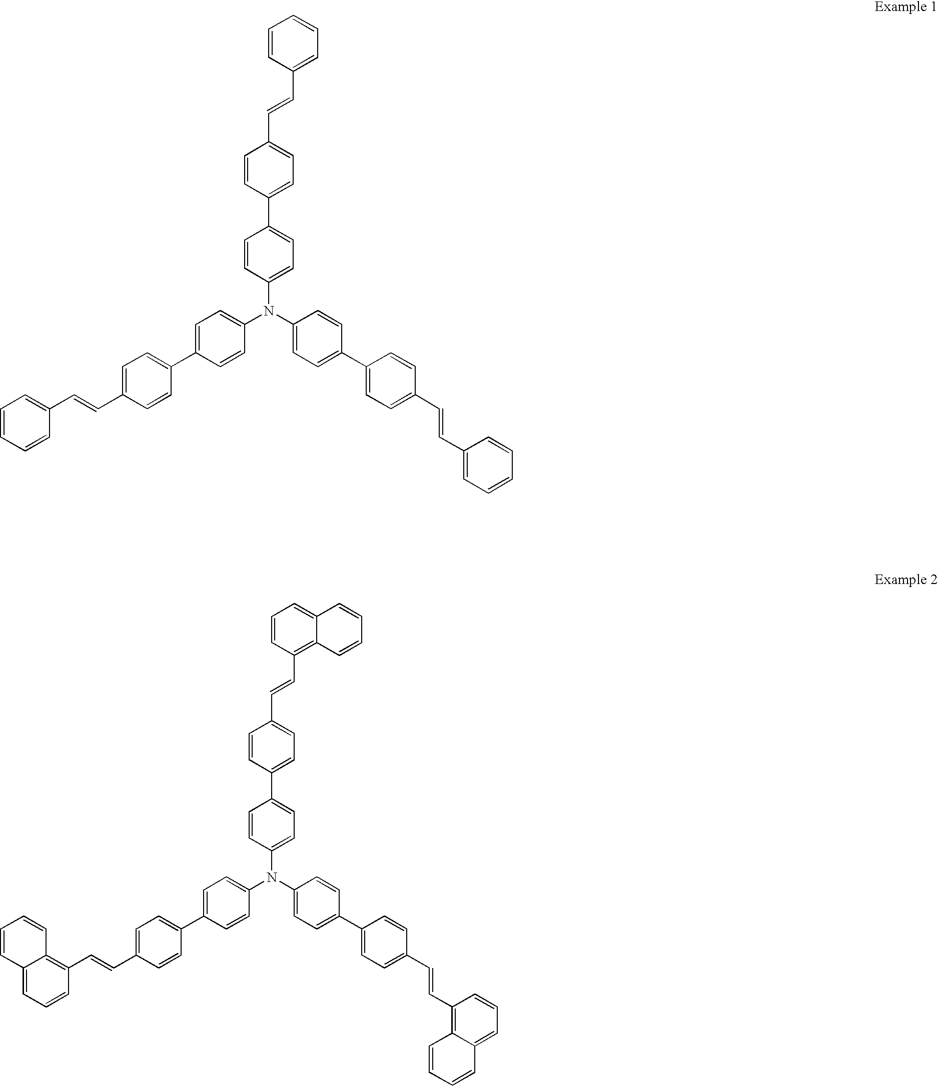

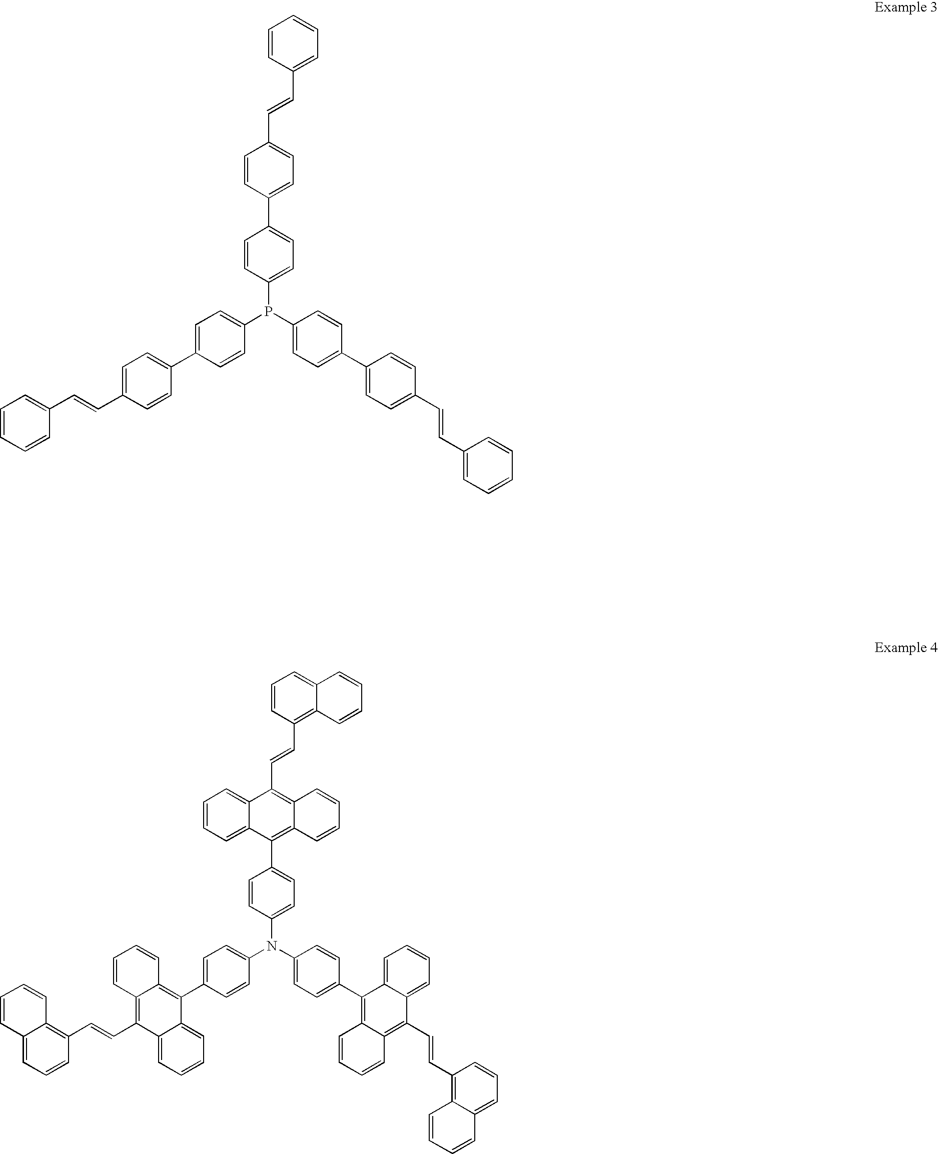

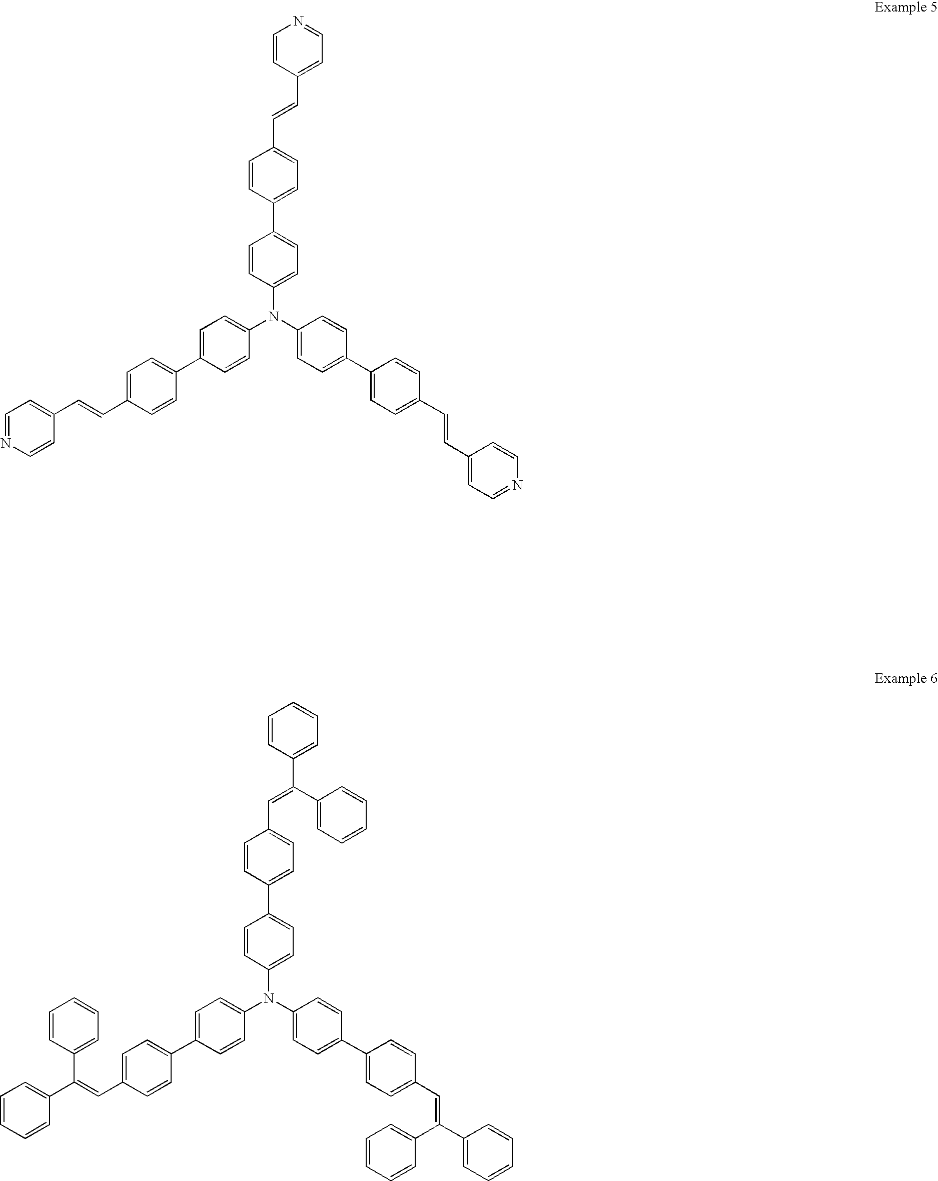

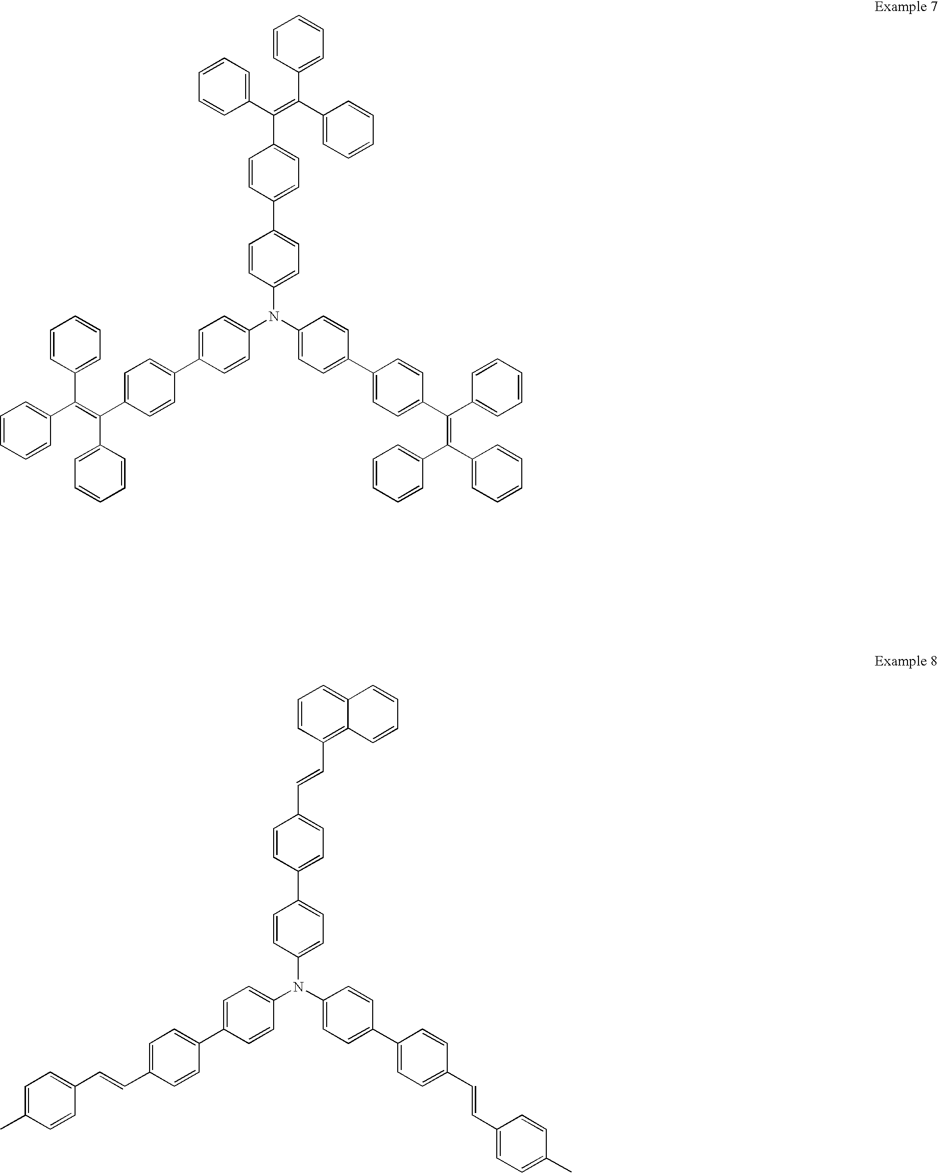

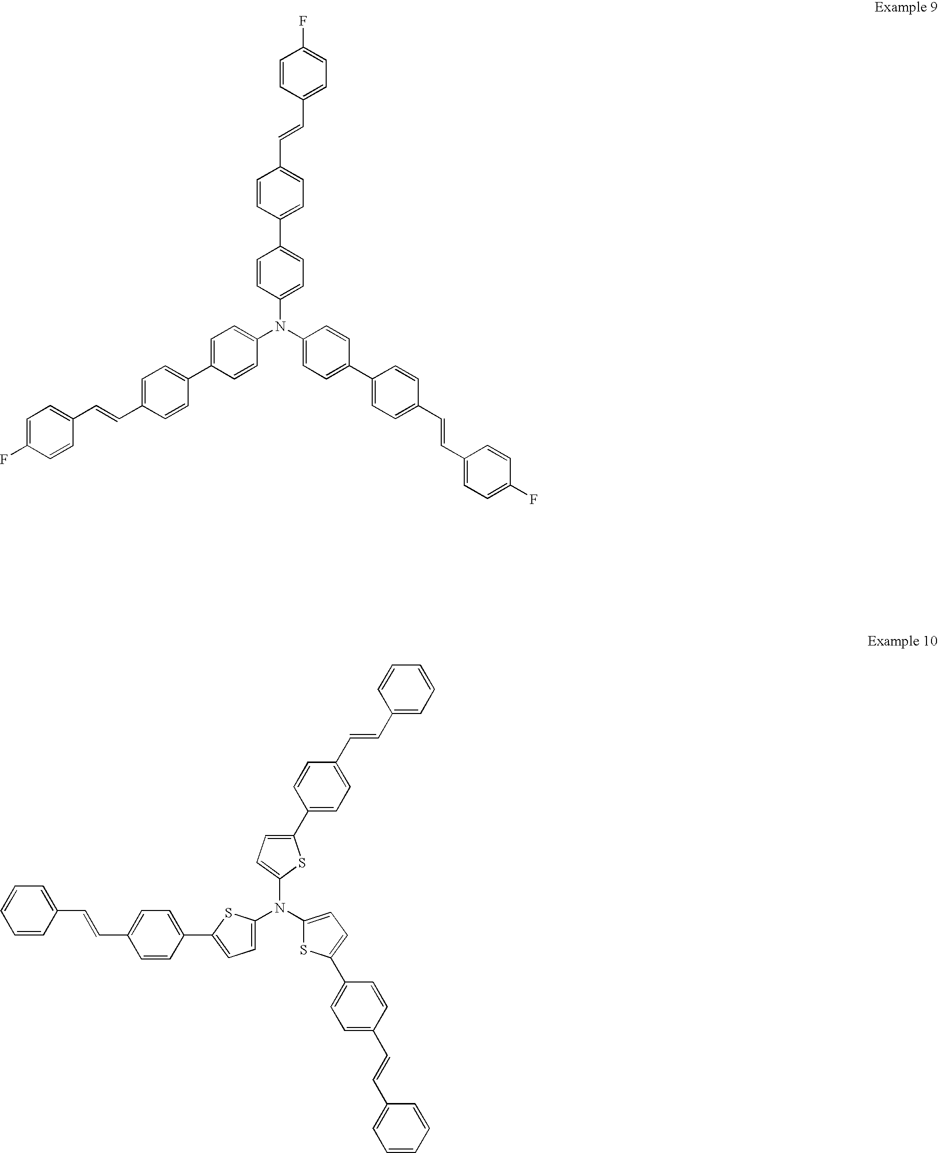

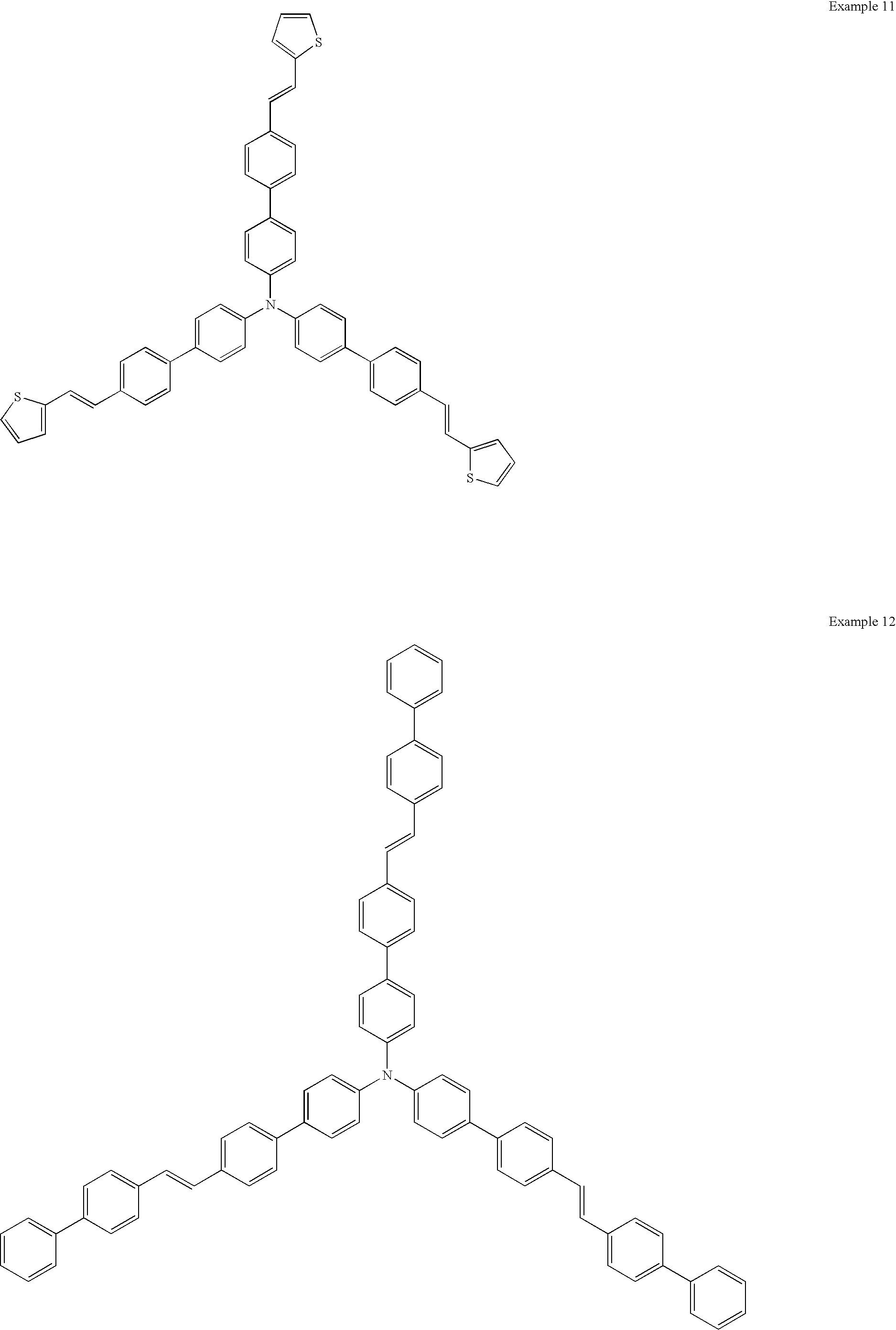

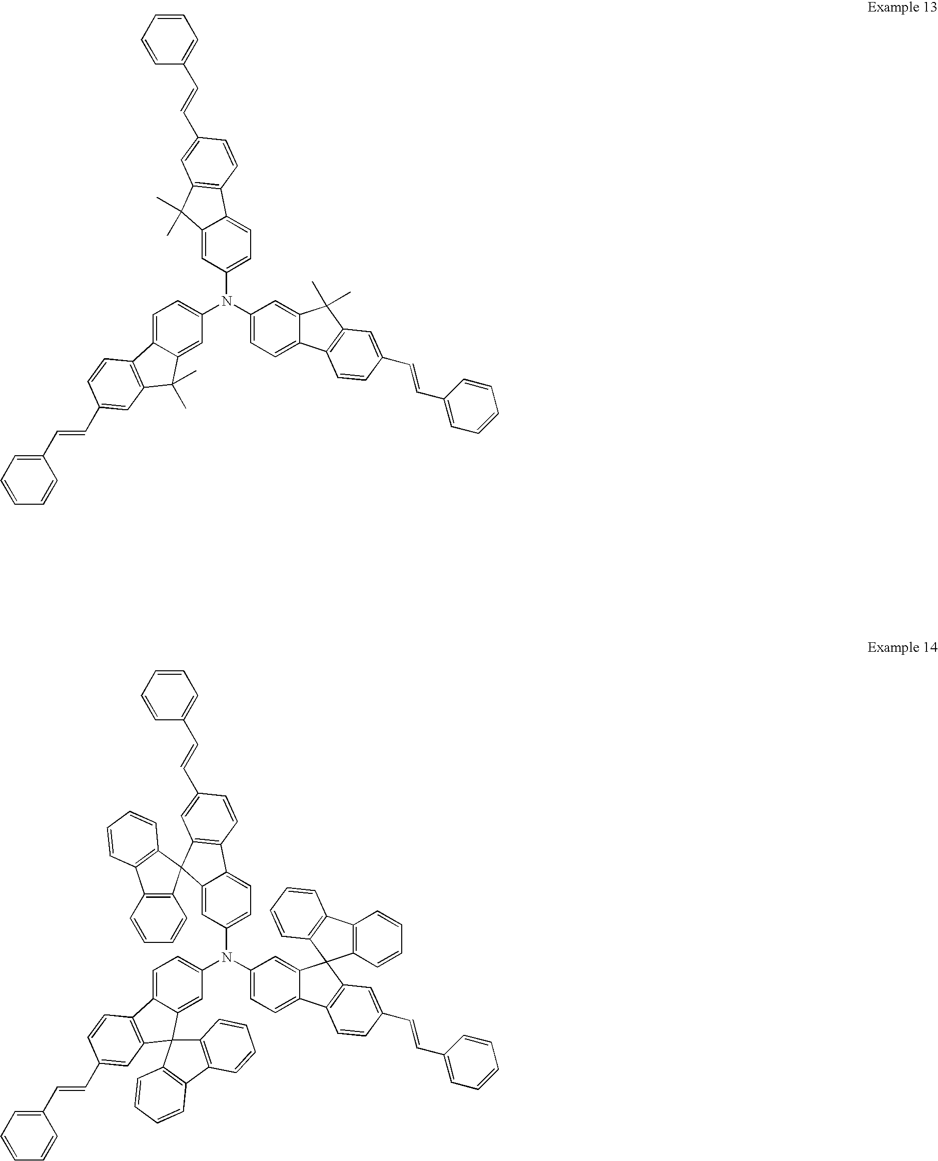

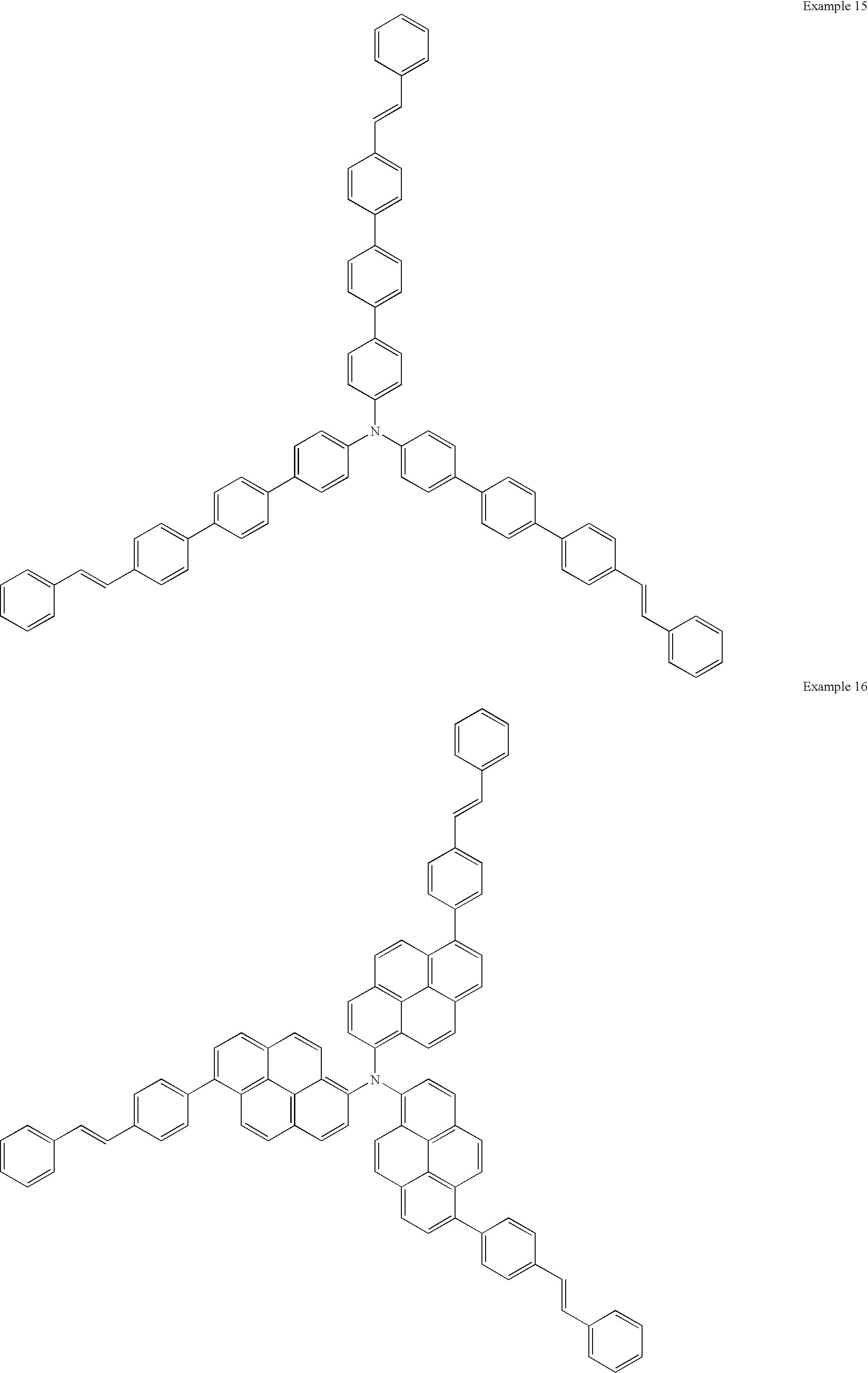

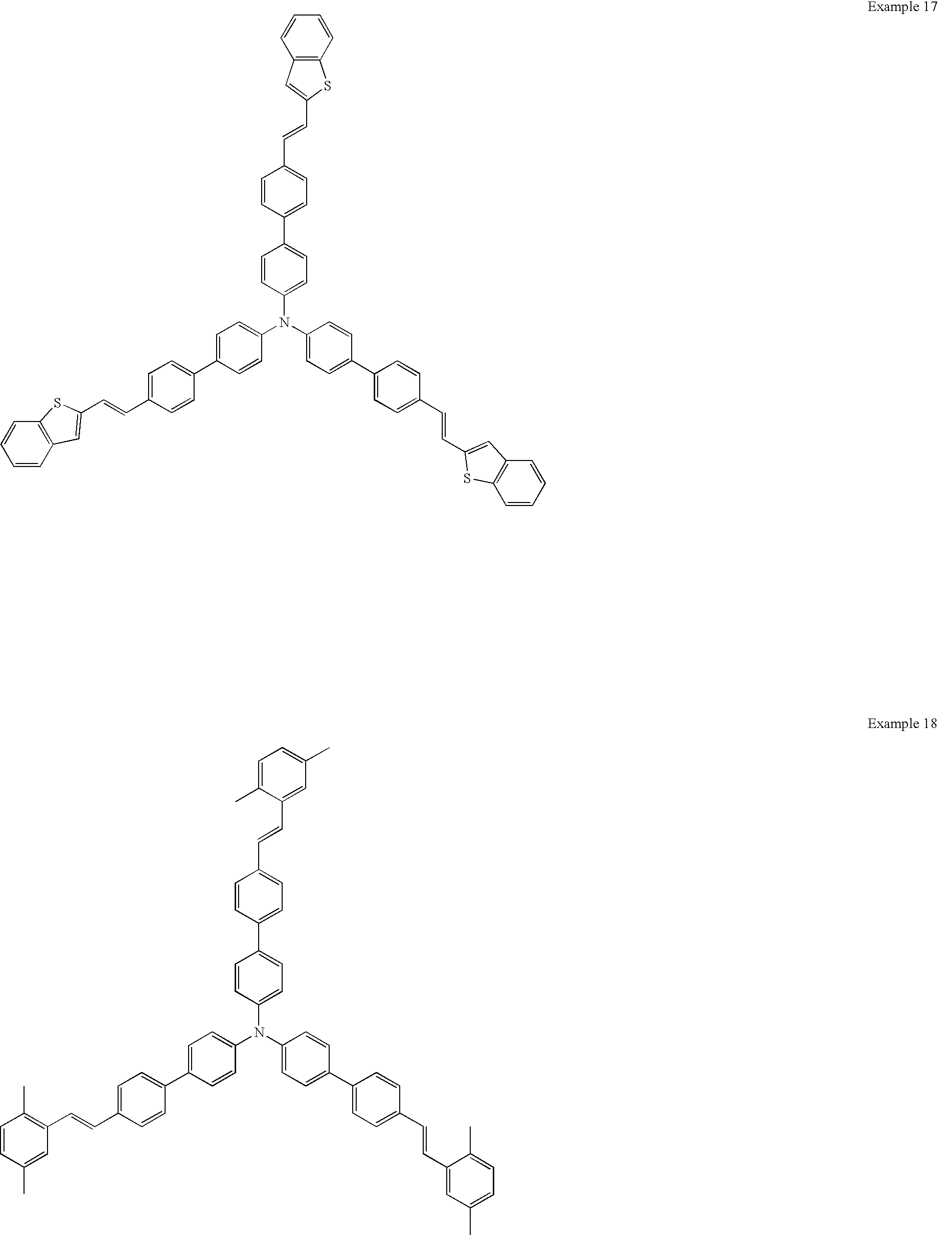

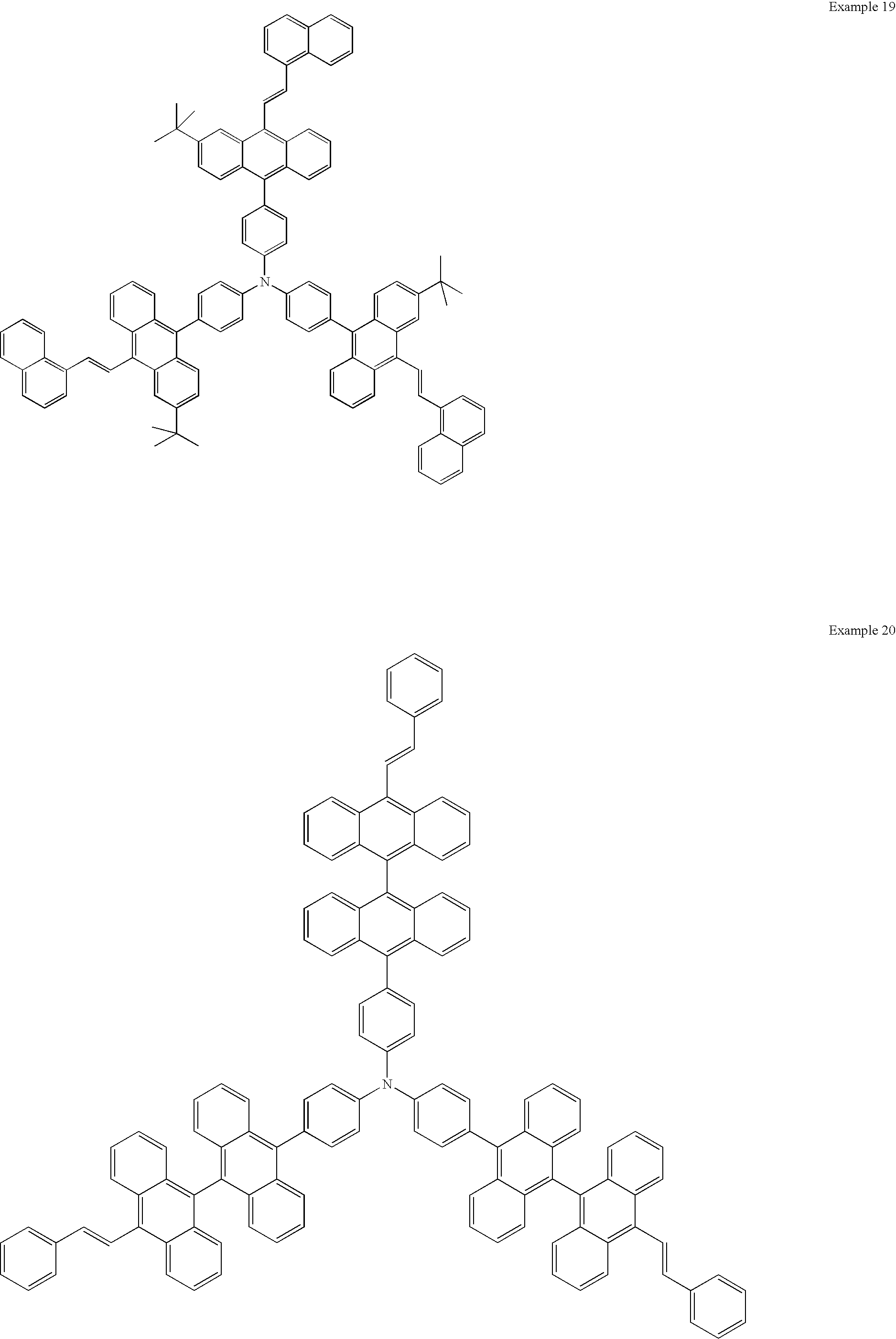

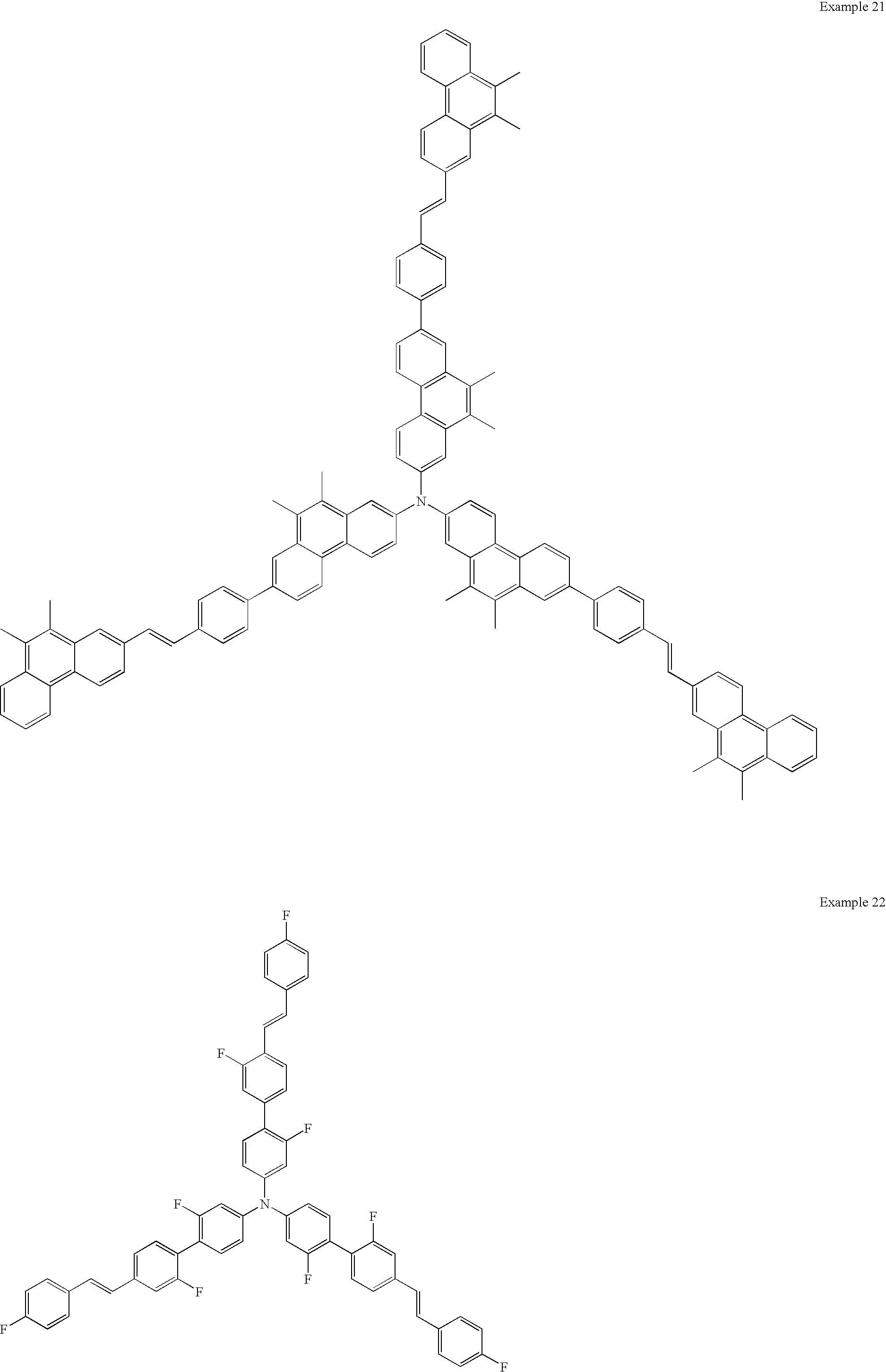

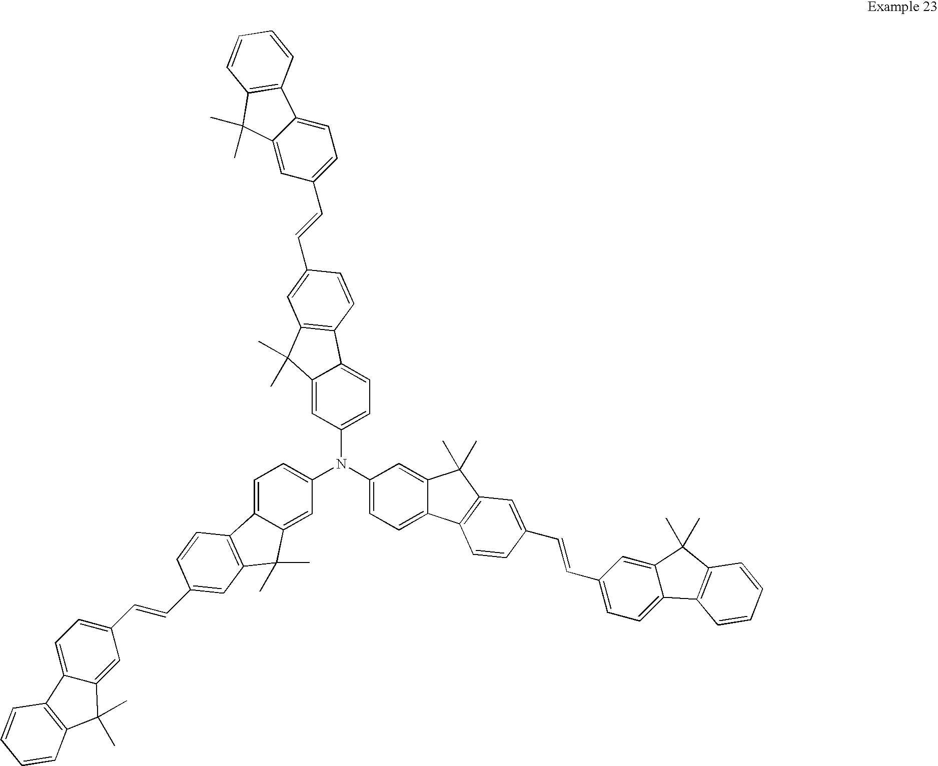

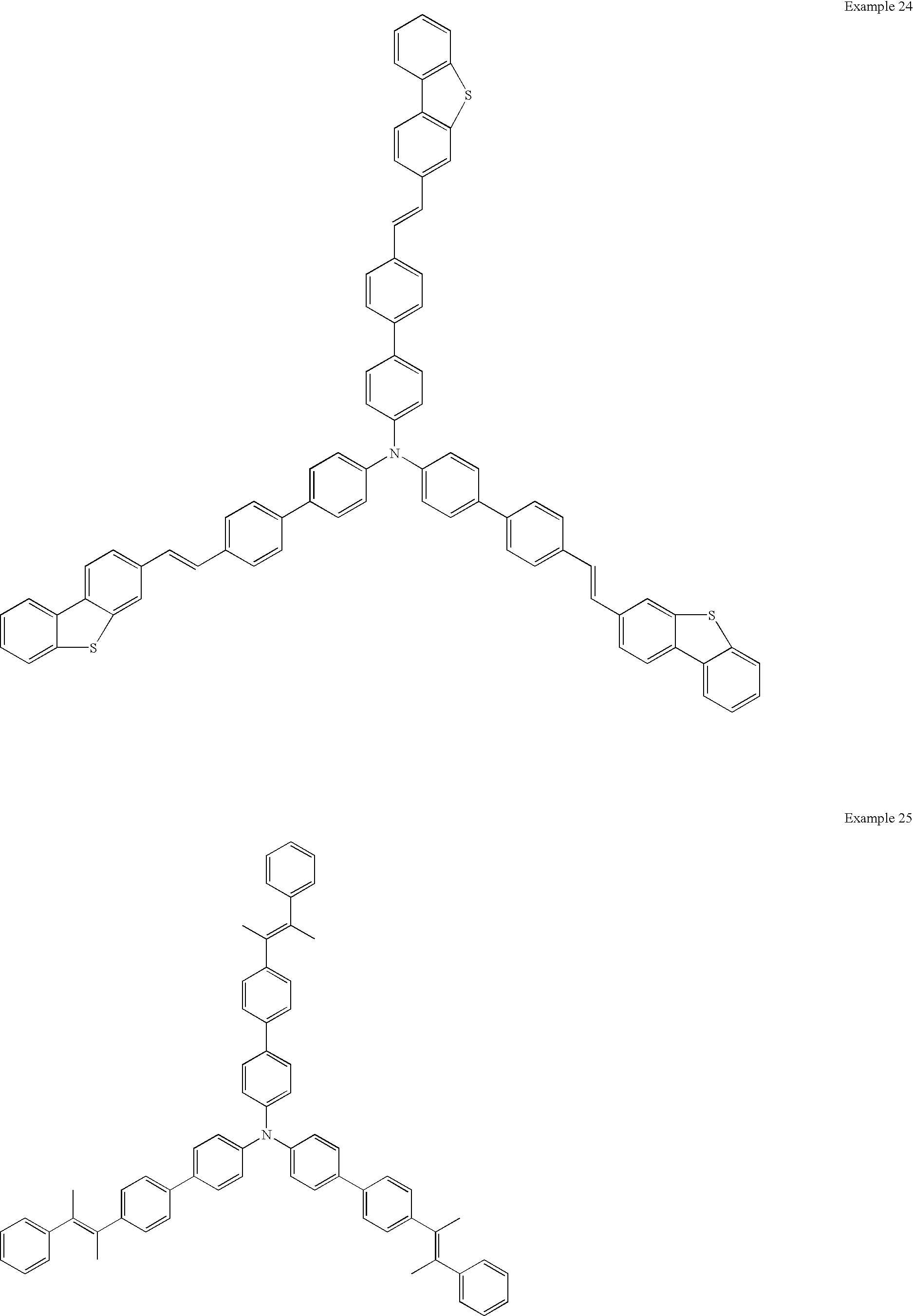

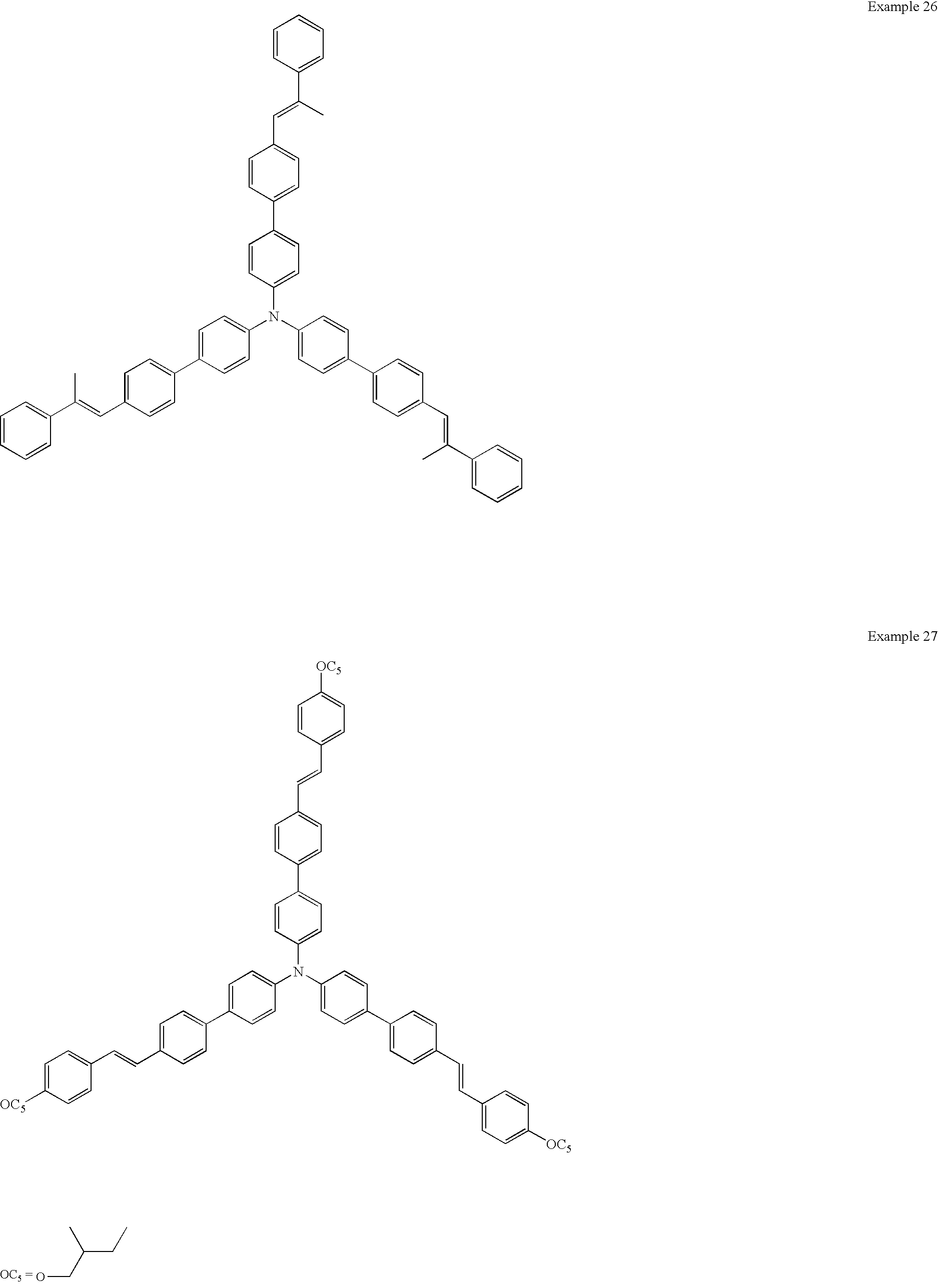

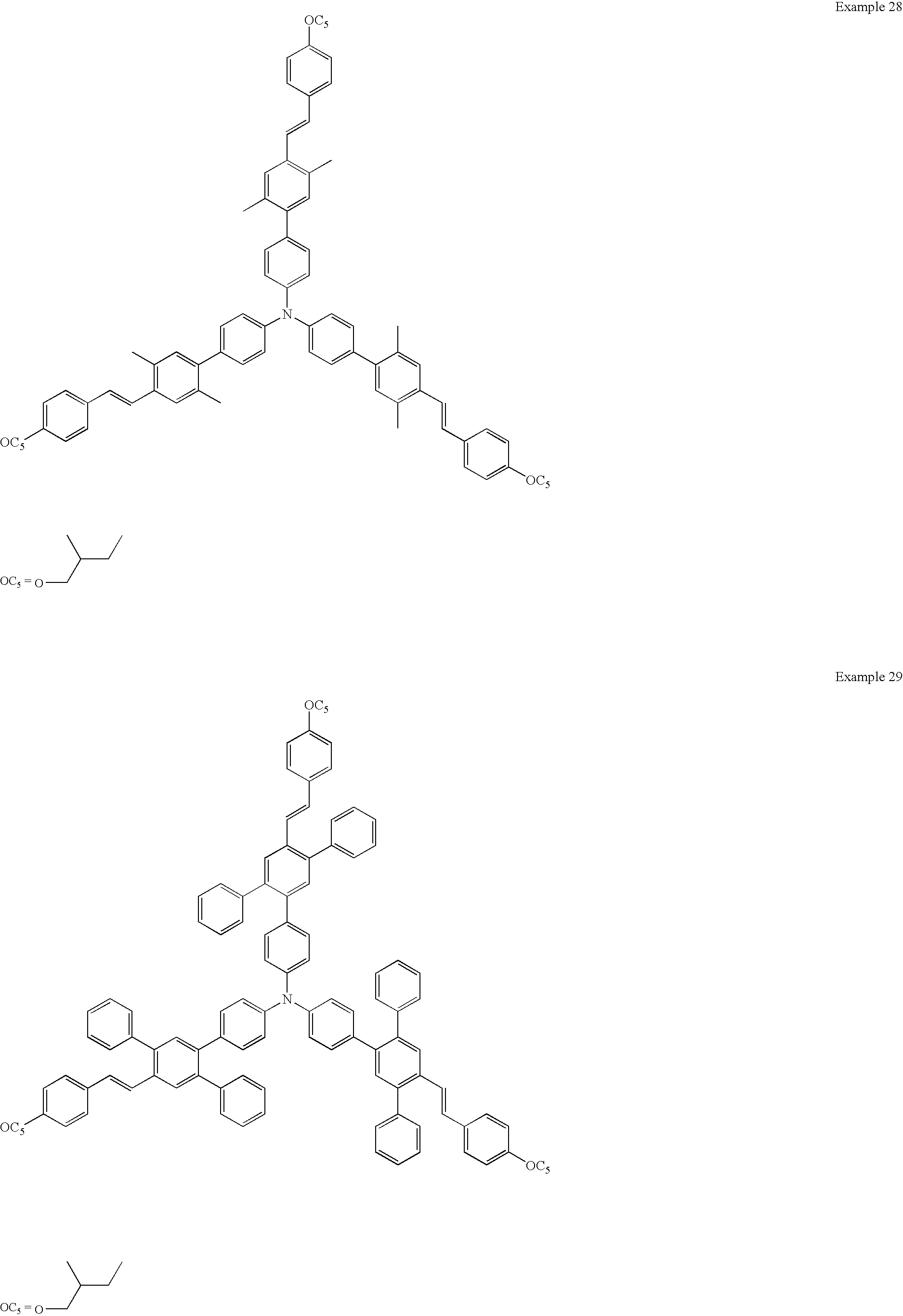

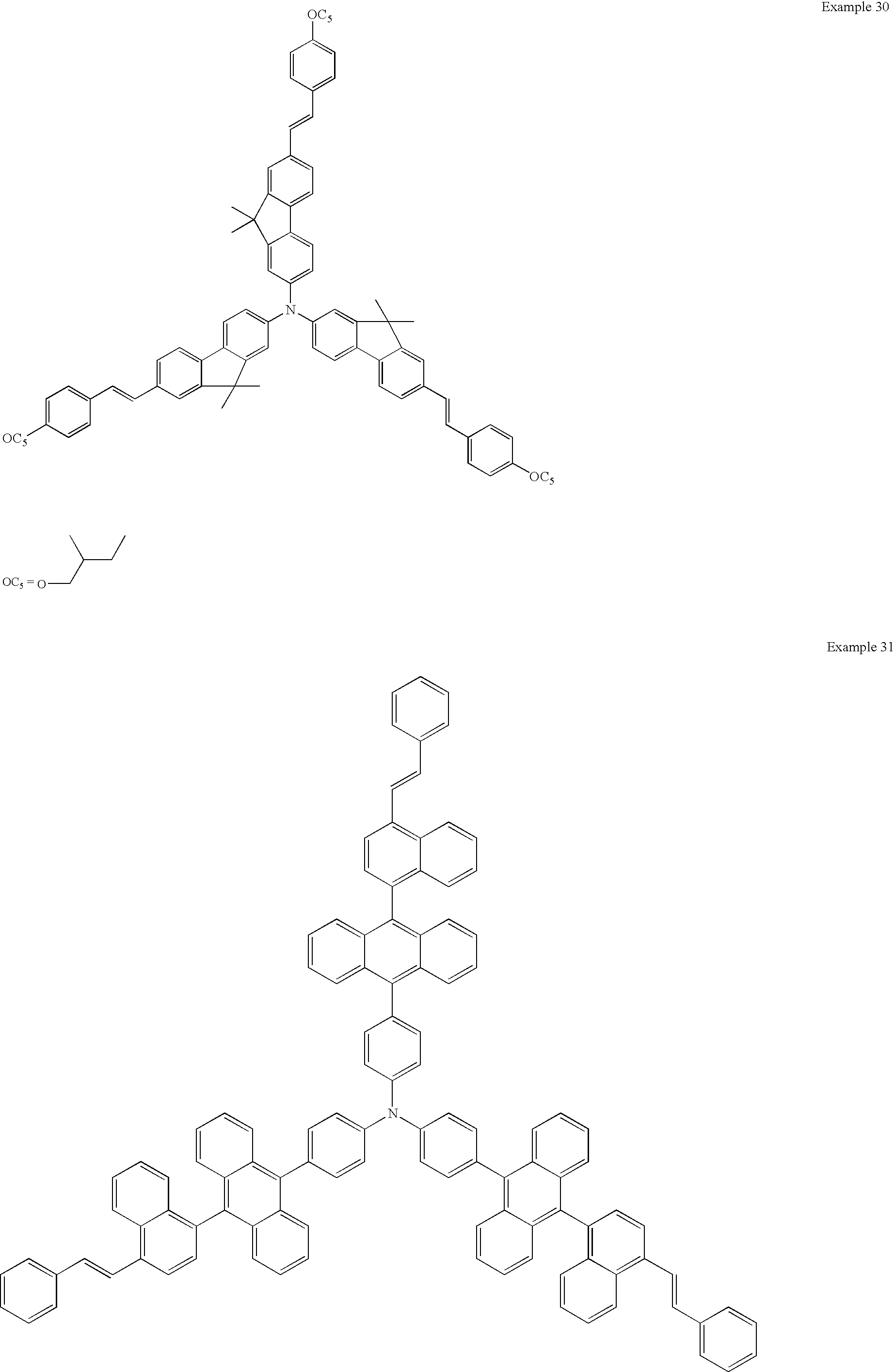

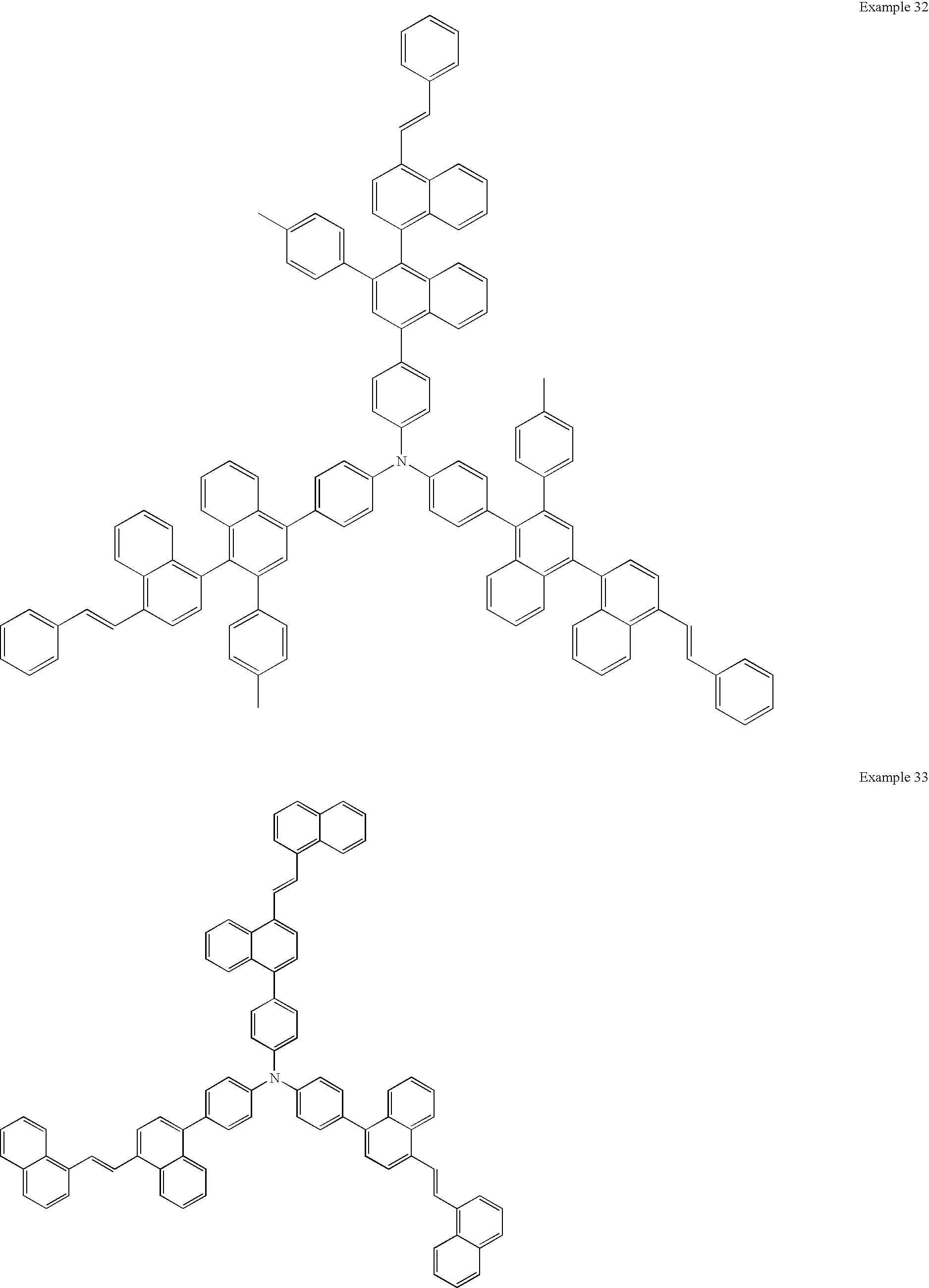

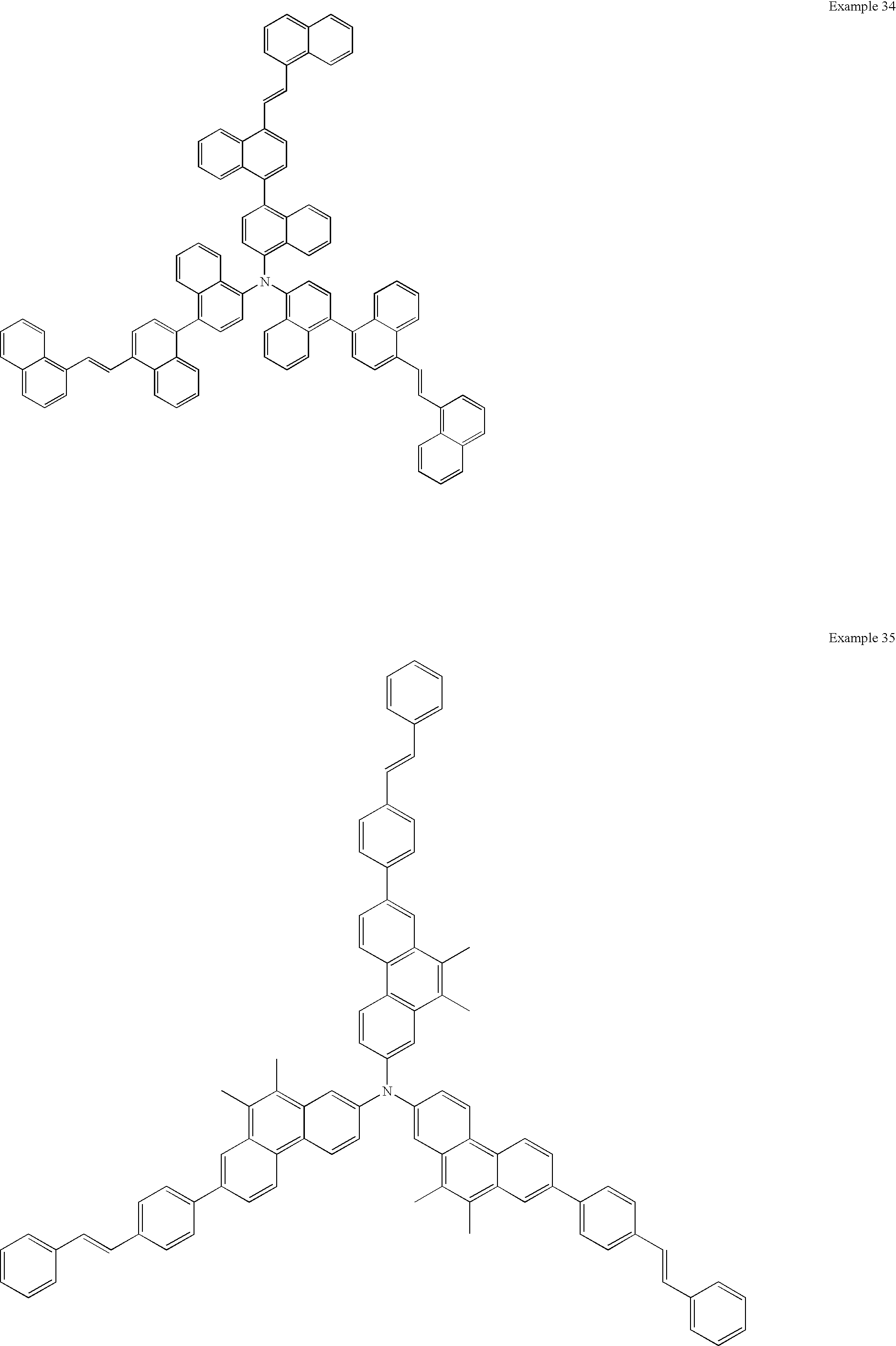

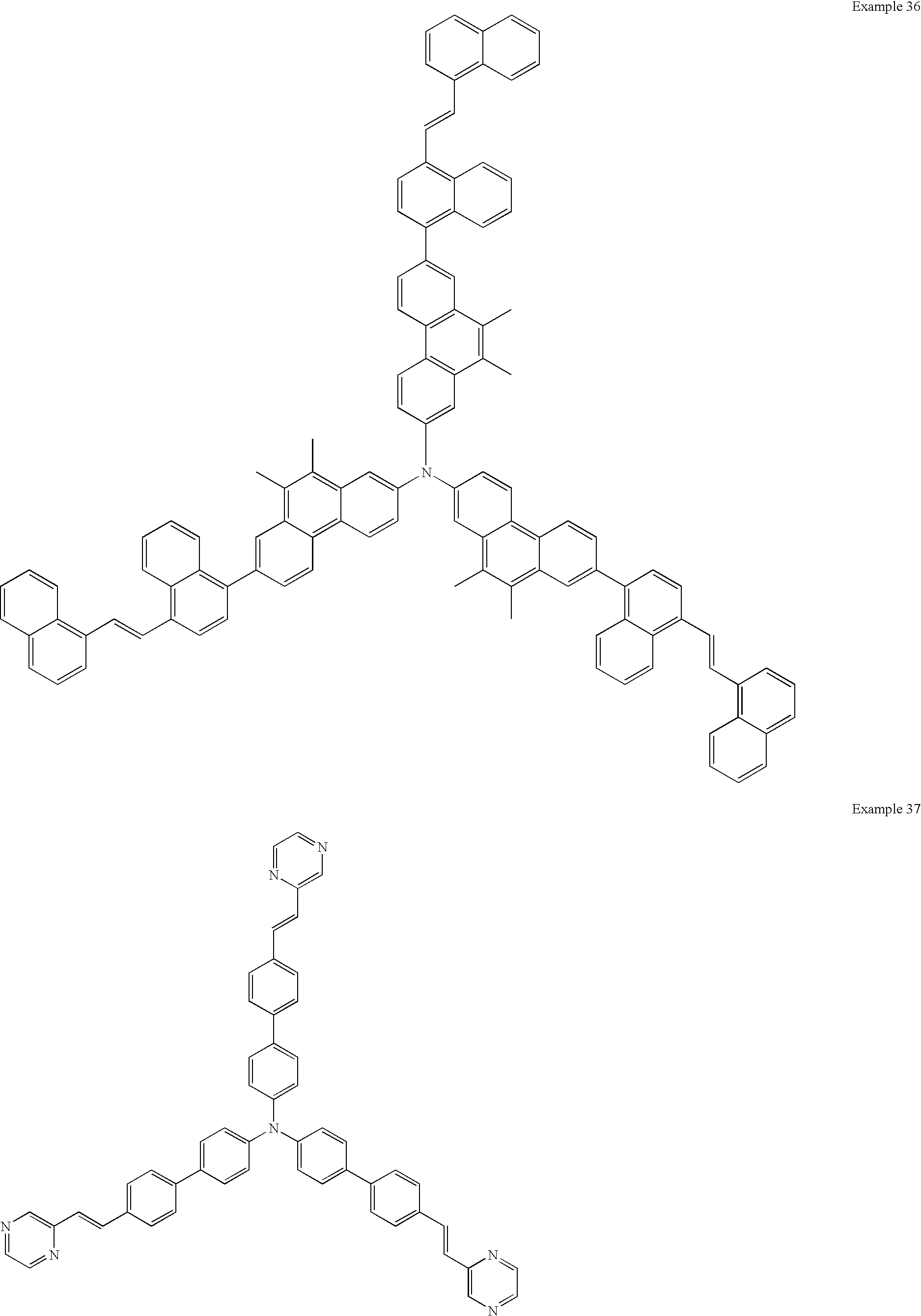

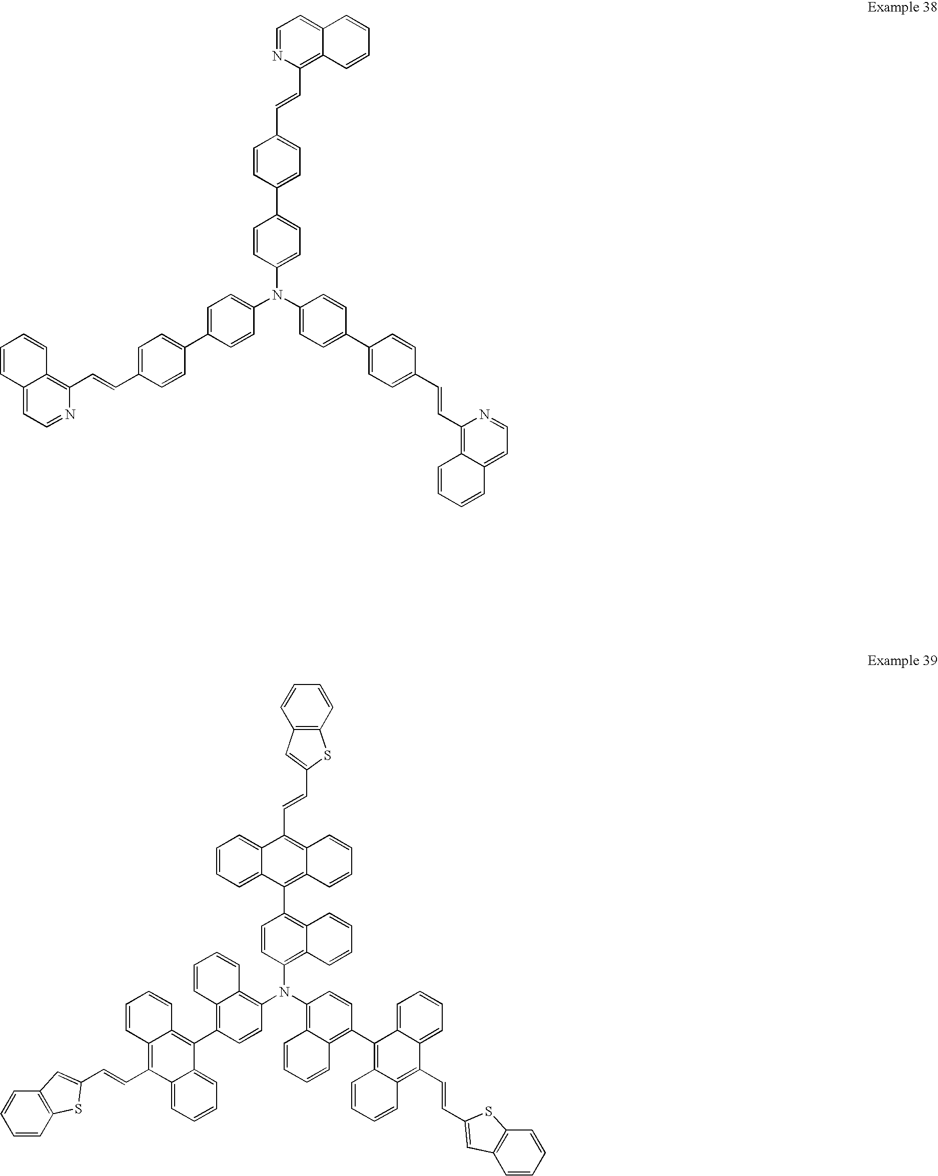

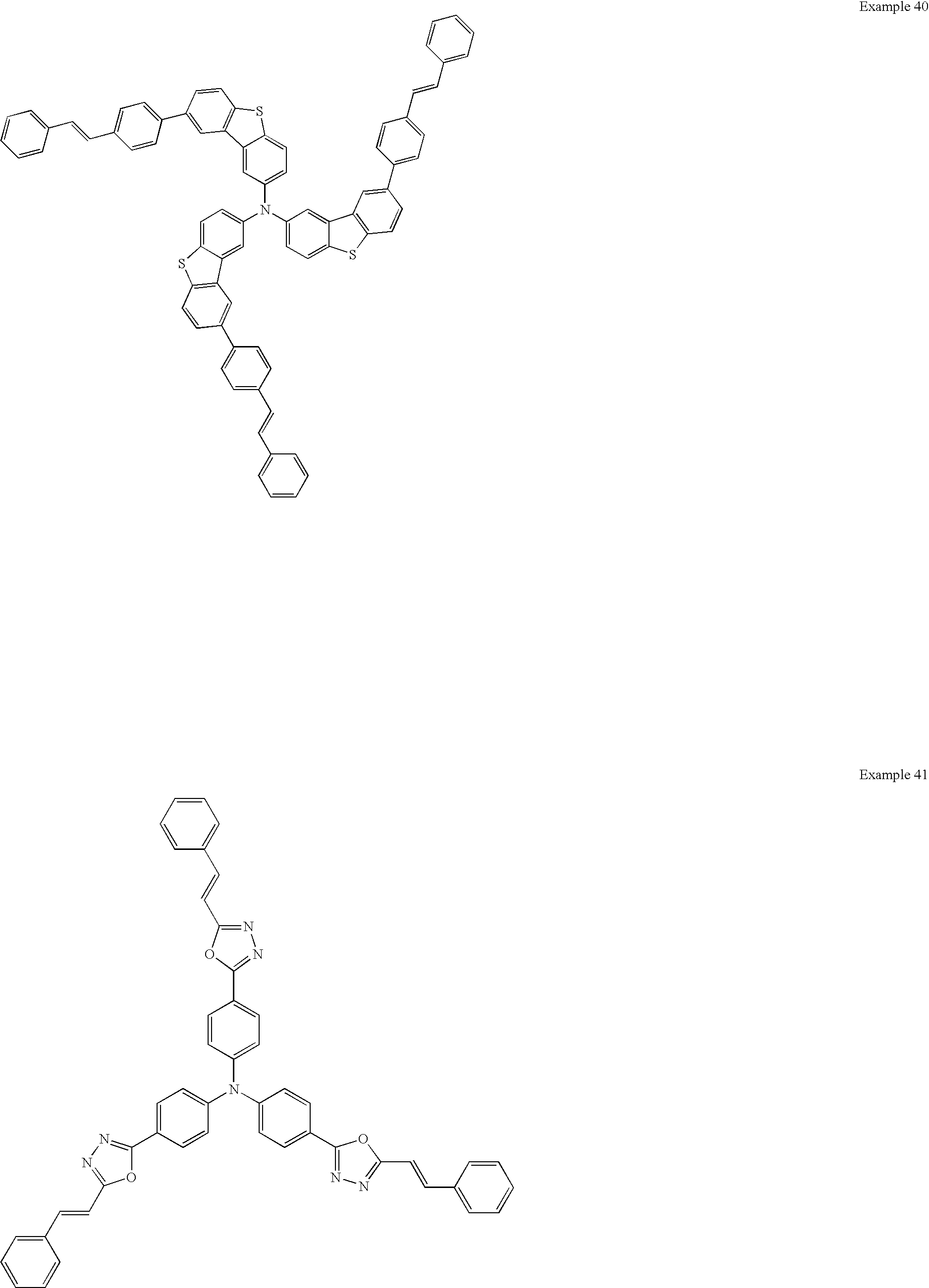

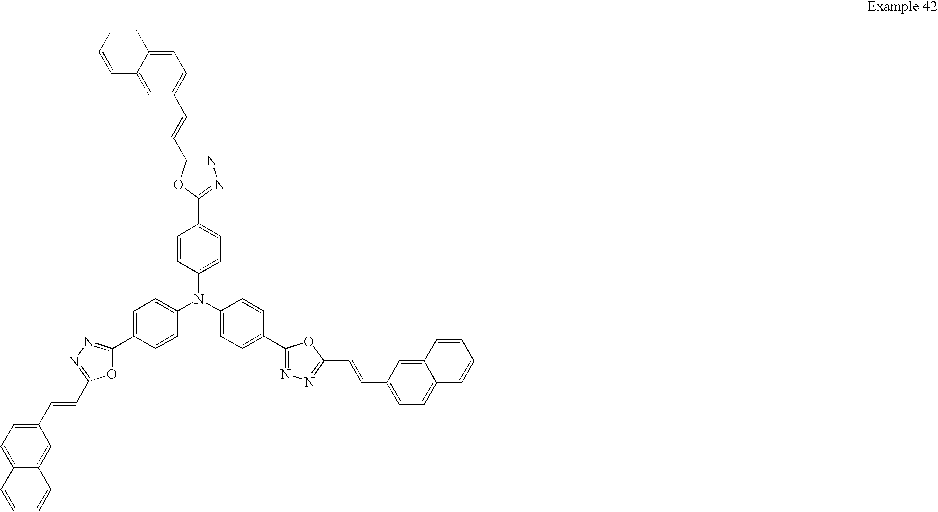

The compounds according to the invention can be prepared by synthetic steps known to the person skilled in the art, such as, for example, bromination, Suzuki coupling, Wittig-Horner reaction, etc. Thus, the bromination of readily available triarylamines gives tris-p-bromine-substituted triarylamines, where—due to the strong +M-directed effect of the nitrogen atom—very good yields at the same time as excellent regioselectivities are frequently achieved here. The brominating agent used, besides elemental bromine, can also be, in particular, N-bromo compounds, such as N-bromosuccinimide (NBS). The tris-p-bromine-substituted triarylamines prepared in this way can easily be reacted with functionalised arylboronic acids, for example by Suzuki coupling under standard conditions, in excellent yields. Suitable functionalisation are, in particular, formyl, alkylcarbonyl and arylcarbonyl groups or protected analogues thereof, for example in the form of the corresponding dioxolanes. It is of course also possible to use other coupling reactions (for example Stille coupling, etc.). The carbonyl substrates obtained in this way can then easily be converted into the corresponding olefins, for example by a Wittig-Horner reaction.

The compounds of the formula (1) can be employed in organic electroluminescent devices. In this case, the compound is preferably employed in the emitting layer as a mixture with at least one host material. It is preferred for the compound of the formula (1) to be the emitting compound (the dopant) in the mixture. Preferred host materials are organic compounds whose emission is of shorter wavelength than that of the compound of the formula (1) or which do not emit at all in the visible region. It is also possible to use the compounds of the formula (1) as hole-transport material.

Suitable host materials are various classes of substance. Preferred host materials are selected from the classes of the oligoarylenes (for example 2, 2′,7,7′-tetraphenyl-spirobifluorene as described in EP 676461 or dinaphthylanthracene), in particular the oligoarylenes containing condensed aromatic groups, the oligoarylenevinylenes (for example DPVBi or spiro-DPVBi as described in EP 676461), the polypodal metal complexes (for example as described in WO 04/081017), the hole-conducting compounds (for example as described in WO 04/058911), the electron-conducting compounds, in particular ketones, phosphine oxides, sulfoxides, etc. (for example as described in the unpublished application DE 102004008304.5) or the atropisomers (for example as described in the unpublished application EP 04026402.0). Particularly preferred host materials are selected from the classes of the oligoarylenes containing naphthalene, anthracene and/or pyrene or atropisomers of these compounds, the oligoarylenevinylenes, the ketones, the phosphine oxides and the sulfoxides. Very particularly preferred host materials are selected from the classes of the oligoarylenes containing anthracene and/or pyrene or atropisomers of these compounds, the phosphine oxides and the sulfoxides.

The proportion of the compound of the formula (1) in the mixture is between 0.1 and 99.0% by weight, preferably between 0.5 and 50.0% by weight, particularly preferably between 1.0 and 20.0% by weight, in particular between 1.0 and 10.0% by weight. Correspondingly, the proportion of the host material in the mixture is between 1.0 and 99.9% by weight, preferably between 50.0 and 99.5% by weight, particularly preferably between 80.0 and 99.0% by weight, in particular between 90.0 and 99.0% by weight.

Preference is furthermore given to organic electroluminescent devices, characterised in that a plurality of emitting compounds are used in the same layer or in different layers, where at least one of these compounds has a structure of the formula (1). These compounds particularly preferably have in total a plurality of emission maxima between 380 nm and 750 nm, resulting overall in white emission, i.e. at least one further emitting compound which is able to fluoresce or phosphoresce and emits yellow, orange or red light is used in addition to the compound of the formula (1). Particular preference is given to three-layer systems, where at least one of these layers comprises a compound of the formula (1) and where the layers exhibit blue, green and orange or red emission (for the basic structure, see, for example, WO 05/011013).

Apart from the cathode, anode and emitting layer, the organic electroluminescent device may also comprise further layers. These can be, for example: hole-injection layer, hole-transport layer, electron-transport layer and/or electron-injection layer. However, it should be pointed out at this point that each of these layers does not necessarily have to be present. Thus, in particular on use of compounds of the formula (1) with electron-conducting host materials, very good results are furthermore obtained if the organic electroluminescent device does not comprise a separate electron-transport layer and the emitting layer is directly adjacent to the electron-injection layer or to the cathode. Alternatively, the host material may also simultaneously serve as electron-transport material in an electron-transport layer. It may likewise be preferred for the organic electroluminescent device not to comprise a separate hole-transport layer and for the emitting layer to be directly adjacent to the hole-injection layer or to the anode. It may furthermore be preferred for the compound of the formula (1) not to be used as dopant or not only as dopant in the emitting layer, but instead also as hole-conducting compound (as the pure substance or as a mixture) in a hole-transport layer.

Preference is furthermore given to an organic electroluminescent device, characterised in that one or more layers are coated by a sublimation process, in which the materials are vapour-deposited in vacuum sublimation units at a pressure below 10⁻⁵ mbar, preferably below 10⁻⁶ mbar, particularly preferably below 10⁻⁷ mbar.

Preference is likewise given to an organic electroluminescent device, characterised in that one or more layers are coated by the OVPD (organic vapour phase deposition) process or with the aid of carrier-gas sublimation, in which the materials are applied at a pressure between 10⁻⁵ mbar and 1 bar.

Preference is furthermore given to an organic electroluminescent device, characterised in that one or more layers are produced from solution, such as, for example, by spin coating, or by means of any desired printing process, such as, for example, screen printing, flexographic printing or offset printing, but particularly preferably LITI (light induced thermal imaging, thermal transfer printing) or ink-jet printing. For this purpose, soluble compounds of the formula (1) are necessary. High solubility can be achieved either by suitable substitution of the compounds or alternatively through the choice of suitable atropisomers.

The compounds according to the invention have the following surprising advantages over the prior art on use in organic electroluminescent devices:

1. The efficiency of corresponding devices becomes higher compared with systems in accordance with the prior art, in particular compared with systems which contain only one instead of, in accordance with the invention, two or more aryl or heteroaryl groups between the group Y and the vinyl group.
2. The stability of corresponding devices becomes higher compared with systems in accordance with the prior art, which is evident, in particular, from a significantly longer lifetime.
3. The emission colour of the compounds is darker blue compared with the styryl-amines in accordance with the prior art that are usually used. They are thus more suitable for use in high-quality full-colour displays.
4. The compounds can be sublimed and vapour-deposited well and without significant decomposition, are thus easier to process and are therefore more suitable for use in OLEDs than materials in accordance with the prior art.

The present application text and also the examples below are directed to the use of compounds according to the invention in relation to OLEDs and the corresponding displays. In spite of this restriction of the description, it is possible for the person skilled in the art, without further inventive step, also to use the compounds according to the invention for further uses in other electronic devices, for example for organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs) or also organic laser diodes (O-lasers), to mention but a few applications.

The present invention likewise relates to the use of the compounds according to the invention in the corresponding devices and to these devices themselves.

The invention is explained in greater detail by the following examples without wishing it to be restricted thereby.

### EXAMPLES

The following syntheses were carried out under a protective-gas atmosphere, unless indicated otherwise. The starting materials were purchased from ALDRICH or ABCR (tris(4-bromophenyl)amine, 4-formylbenzeneboronic acid, palladium(II) acetate, tri-o-tolylphosphine, inorganics, solvents).

### Example 1

### Synthesis of tris-1-[(1′-styryl)-4,4′-biphenyl]amine

### a) Synthesis of tris-1-(1′-formyl-4,4′-biphenyl)amine

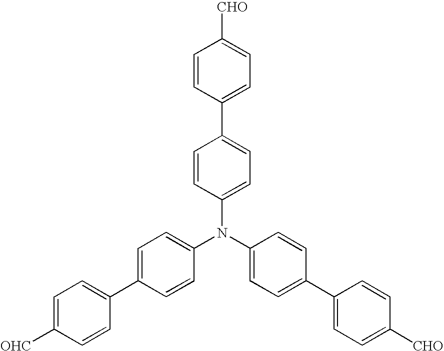

5.5 g (18 mmol) of tri-o-tolylphosphine and then 674 mg (3 mmol) of palladium(II) acetate were added with vigorous stirring to a degassed suspension of 48.2 g (100 mmol) of tris(4-bromophenyl)amine, 67.4 g (450 mmol) of 4-formylbenzene-boronic acid and 156.4 g (630 mmol) of potassium phosphate hydrate in a mixture of 130 ml of dioxane, 300 ml of toluene and 375 ml of water. The mixture was boiled under reflux for 5 h and allowed to cool. The yellow-green precipitate was filtered off with suction, washed three times with 200 ml of ethanol/water (1:1, v:v) and three times with 100 ml of ethanol and subsequently dried in vacuo. Yield: 51.9 g (93 mmol), 93.0% of theory, purity according to ¹H-NMR about 98%.

¹H-NMR (CDCl₃): δ [ppm]=10.05 (s, 3H, CHO), 7.95 and 7.76 (2×d, ³J_{HH}=8.3 Hz, 12H), 7.61 and 7.28 (2×d, ³J_{HH}=8.3 Hz, 12H).

### b) Synthesis of tris-1-[(1′-styryl)-4,4′-biphenyl]amine

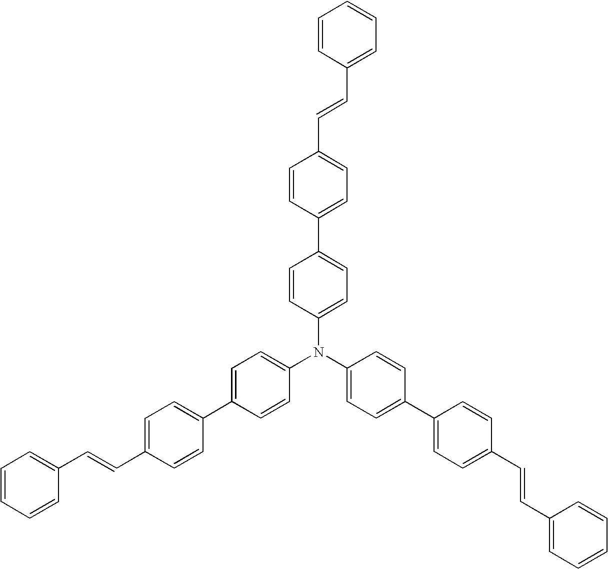

46.1 g (480 mmol) of sodium tert-butoxide were added at 0° C. with vigorous stirring to a mixture of 50.0 ml (240 mmol) of diethyl phenylmethanephosphonate and 1000 ml of DMF. A solution at 30° C. of 37.0 g (66 mmol) of tris-1-(1′-formyl-4,4′-biphenyl)-amine in 1500 ml of DMF was slowly added dropwise to this mixture at 0-10° C. When the addition was complete, the mixture was stirred at 0° C. to 10° C. for a further 3 h and at room temperature for 12 h. 100 ml of 2.5N HCl, then 300 ml of water and then 300 ml of ethanol were subsequently added. The yellow microcrystalline precipitate was filtered off with suction (P3), washed three times with 200 ml of a mixture of ethanol/water (1:1, v:v) each time and three times with 200 ml of ethanol each time. After drying in vacuo, the solid was recrystallised six times from DMF (about 20 ml/g) with exclusion of light. Yield: 27.2 g (35 mmol), 52.8%, purity according to HPLC>99.9%.

¹H-NMR (tetrachloroethane-d2): δ [ppm]=7.64-7.53 (m, 24H), 7.39-7.36 (m, 6H), 7.29-7.24 (m, 9H), 7.15 (br. s, 6H).

### Example 2

### Production of OLEDs

OLEDs were produced by a general process as described in WO 04/058911, which was adapted in individual cases to the respective circumstances (for example layer-thickness variation in order to achieve optimum efficiency or colour).

The results for various OLEDs are presented in the following examples. The basic structure, the materials and layer thicknesses used, apart from the emitting layer and the hole-transport layer, were identical for better comparability. OLEDs having the following structure were produced analogously to the above-mentioned general process:
Hole-injection layer (HIL) 20-600 nm PEDOT/PSS (spin-coated from aqueous dispersion; purchased from H.C. Starck, Goslar, Germany; poly(3,4-ethylenedioxy-2,5-thiophene)+poly-styrenesulfonic acid)
Hole-transport layer (HTM) precise structure see Table 1 NaphDATA (vapour-deposited; purchased from SynTec, Wolfen, Germany; 4,4′,4″-tris(N-1-naphthyl-N-phenylamino)triphenylamine) and/or S-TAD (vapour-deposited; prepared as described in WO 99/12888; 2,2′,7,7′-tetrakis(diphenylamino)spiro-9,9′-bifluorene) and/or 20 nm NPB (vapour-deposited; N-naphthyl-N-phenyl-4,4′-diaminobiphenyl) and/or 20 nm HTM1 (vapour-deposited; prepared as described in WO 99/12888; 2,2′,7,7′-tetrakis(di-para-tolylamino)-spiro-9,9′-bifluorene)
Emission layer (EML) see Table 1 for materials, concentration and layer thicknesses
Electron conductor (ETL) 20 nm AlQ₃ (purchased from SynTec; tris(quinolinato)-aluminium(III))
LiF—Al (cathode) 1 nm LiF, 150 nm Al on top

These still unoptimised OLEDs were characterised by standard methods; for this purpose, the electroluminescence spectra, the efficiency (measured in cd/A), the power efficiency (measured in Im/W) as a function of the brightness, calculated from current/voltage/luminous density characteristic lines (IUL characteristic lines), and the lifetime were determined. The lifetime is defined as the time after which the initial brightness of 500 cd/m² has dropped to half.

Table 1 shows the results for some OLEDs (Examples 3 to 6), with the composition of the EML and HTL including the layer thicknesses also being shown in each case. Example 3 shows comparative examples which comprise in the emission layer either only various host materials usually used or dopant D2 in accordance with the above-mentioned prior art in a host material. In Examples 4 to 6 according to the invention, the EMLs comprise dopant D1 (as described in Example 1) as emitting materials of the formula (1). Compounds H1 to H3 depicted below serve as host materials. In each case two of the above-mentioned materials NaphDATA, S-TAD, NPB and HTM1, which are applied in two layers one above the other, serve as hole-transport materials. For better clarity, the corresponding structural formulae of the dopants and host materials used are depicted below:

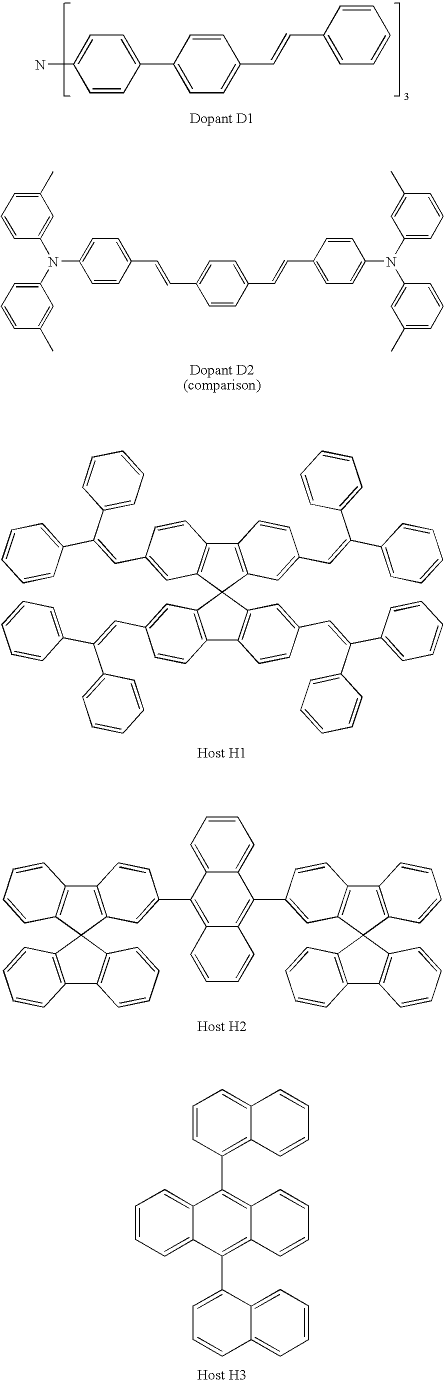

TABLE 1

| | | | | Max. | Voltage | | |
|---|---|---|---|---|---|---|---|
| | | | | efficiency | (V) at | | |
| Example | HTL1 | HTL2 | EML | (cd/A) | 100cd/m² | CIE ^{a} | Lifetime (h) ^{b} |
| Example 3a | NaphDATA | S-TAD | H1 | 4.2 | 5.8 | x = 0.17 | 1400 |
| (comparison) | (20 nm) | (20 nm) | (30 nm) | | | y = 0.26 | |
| Example 3b | NaphDATA | S-TAD | H1:D2 (5%) | 4.9 | 6.3 | x = 0.17 | 1100 |
| (comparison) | (20 nm) | (20 nm) | (30 nm) | | | y = 0.31 | |
| Example 3c | NaphDATA | S-TAD | H2 | 3.3 | 6.5 | x = 0.15 | 450 |
| (comparison) | (20 nm) | (20 nm) | (30 nm) | | | y = 0.15 | |
| Example 3d | NaphDATA | S-TAD | H3 | 1.1 | 5.8 | x = 0.17 | 800 |
| (comparison) | (20 nm) | (20 nm) | (30 nm) | | | y = 0.19 | |
| Example 4a | NaphDATA | S-TAD | H3:D1 (5%) | 3.7 | 5.2 | x = 0.15 | 1300 |
| | (20 nm) | (20 nm) | (30 nm) | | | y = 0.10 | |
| Example 4b | NaphDATA | S-TAD | H3:D1 (10%) | 3.6 | 4.7 | x = 0.15 | 1600 |
| | (20 nm) | (20 nm) | (30 nm) | | | y = 0.13 | |
| Example 5 | NaphDATA | NPB | H3:D1 (2%) | 5.2 | 5.3 | x = 0.16 | 900 |
| | (20 nm) | (20 nm) | (30 nm) | | | y = 0.11 | |
| Example 6a | HTM1 | NPB | H3:D1 (2%) | 2.7 | 5.3 | x = 0.16 | 3000 |
| | (20 nm) | (20 nm) | (30 nm) | | | y = 0.10 | |
| Example 6b | HTM1 | NPB | H3:D1 (5%) | 3.2 | 5.1 | x = 0.16 | 4100 |
| | (20 nm) | (20 nm) | (30 nm) | | | y = 0.13 | |

^{a} CIE coordinates: colour coordinates of the Commission Internationale de I'Eclairage 1931.
^{b} Lifetime: time until the brightness has dropped to 50% of the initial brightness, measured at an initial brightness of 500 cd/m².

In summary, it can be stated that OLEDs comprising emitting compounds of the formula (1) have a longer lifetime with a significantly darker blue colour than materials in accordance with the prior art, as can easily be seen from Table 1. These compounds are therefore more suitable for use in OLEDs than materials in accordance with the prior art.

1-20. (canceled)

21. Compounds of the formula (1) 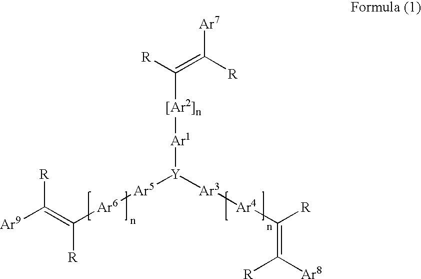
wherein
Y is on each occurrence N, P, As, Sb, P═O, As═O or Sb═O;
Ar¹, Ar², Ar³, Ar⁴, Ar⁵, and Ar⁶ are on each occurrence, identically or differently, a divalent aryl or heteroaryl group having up to 24 C atoms, which is optionally substituted by one or more radicals R¹;
Ar⁷, Ar⁸, and Ar⁹ are on each occurrence, identically or differently, a monovalent aryl or heteroaryl group having up to 24 C atoms, which is optionally substituted by one or more radicals R¹;
R is on each occurrence, identically or differently, H, CN, or a straight-chain, branched or cyclic alkyl group having up to 40 C atoms, which is optionally substituted by one or more radicals R², and wherein one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²—, and wherein one or more H atoms is optionally replaced by F, Cl, Br, I, CN, NO₂, or a monovalent aryl or heteroaryl group having up to 24 C atoms, which is optionally substituted by one or more radicals R¹;
R¹ is on each occurrence, identically or differently, H, F, Cl, Br, I, CN, NO₂, or a straight-chain, branched or cyclic alkyl, alkoxy or thioalkoxy group having up to 40 C atoms, which is optionally substituted by one or more radicals R², wherein one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²—, and wherein one or more H atoms is optionally replaced by F, Cl, Br, I, CN, NO₂, an aryl or heteroaryl group having up to 24 C atoms, which is optionally substituted by one or more radicals R², an aryloxy or heteroaryloxy group having up to 24 aromatic C atoms, which is optionally substituted by one or more radicals R², or a combination of two, three, four or five of these systems; and wherein two or more substituents R¹, on the same ring and/or on different rings, optionally define a monocyclic or polycyclic aliphatic or a monocyclic or polycyclic aromatic ring system with one another;
R² is on each occurrence, identically or differently, H or an aliphatic or aromatic hydrocarbon radical having up to 20 C atoms;
n is on each occurrence, identically or differently, 1, 2, 3, 4 or 5.

22. Compounds according to claim 21, wherein Y is N, P, or P═O.

23. Compounds according to claim 21, wherein Ar¹, Ar², Ar³, Ar⁴, Ar⁵ and Ar⁶ are, identically or differently on each occurrence, a divalent aryl or heteroaryl group having up to 16 C atoms, which is optionally substituted by one or two radicals R¹.

24. Compounds according to claim 21, wherein Ar⁷, Ar⁸ and Ar⁹ are, identically or differently on each occurrence, a monovalent aryl or heteroaryl group having up to 16 C atoms, which is optionally substituted by one or more radicals R¹.

25. Compounds according to claim 21, wherein R is, identically or differently on each occurrence, H, CN, a straight-chain or branched alkyl group having up to 4 C atoms, wherein one or more non-adjacent CH₂ groups is optionally replaced by —R²C═CR²—, —C≡C—, —O— or —S— and wherein one or more H atoms is optionally replaced by F or a monovalent aryl or monovalent heteroaryl group having up to 16 C atoms, which is optionally substituted by one or more radicals R¹.

26. Compounds according to claim 21, wherein R¹ is, identically or differently on each occurrence, H, F, CN, or a straight-chain, branched or cyclic alkyl or alkoxy group having up to 10 C atoms, which is optionally substituted by one or more radicals R², wherein one or more non-adjacent CH₂ groups is optionally replaced by —R²C═CR²—, —C≡C—, Si(R²)₂, C═O, —O— or —S—, and wherein one or more H atoms is optionally replaced by F, an aryl or heteroaryl group having up to 16 C atoms, which is optionally substituted by one or more radicals R², an aryloxy or heteroaryloxy group having up to 16 aromatic C atoms, which is optionally substituted by one or more radicals R², or a combination of two, three or four of these systems; and wherein two or more substituents R¹, on the same ring and/or on different rings, optionally define a monocyclic or polycyclic aliphatic or a monocyclic or polycyclic aromatic ring system with one another.

27. Compounds according to claim 21, wherein n is, identically or differently on each occurrence, 1, 2 or 3.

28. Compounds according to claim 21, wherein Ar¹, Ar³ and Ar⁵ are the same aryl or heteroaryl group.

29. Compounds according to claim 21, wherein Ar², Ar⁴ and Ar⁶ are the same aryl or heteroaryl group.

30. Compounds according to claim 21, wherein Ar⁷, Ar⁸ and Ar⁹ are the same aryl or heteroaryl group.

31. Compounds according to claim 21, wherein said compounds have a symmetrical structure and a three-fold axis of rotation.

32. Compounds according to claim 21, wherein said compounds are selected from the group consisting of example structures 1 to 42:

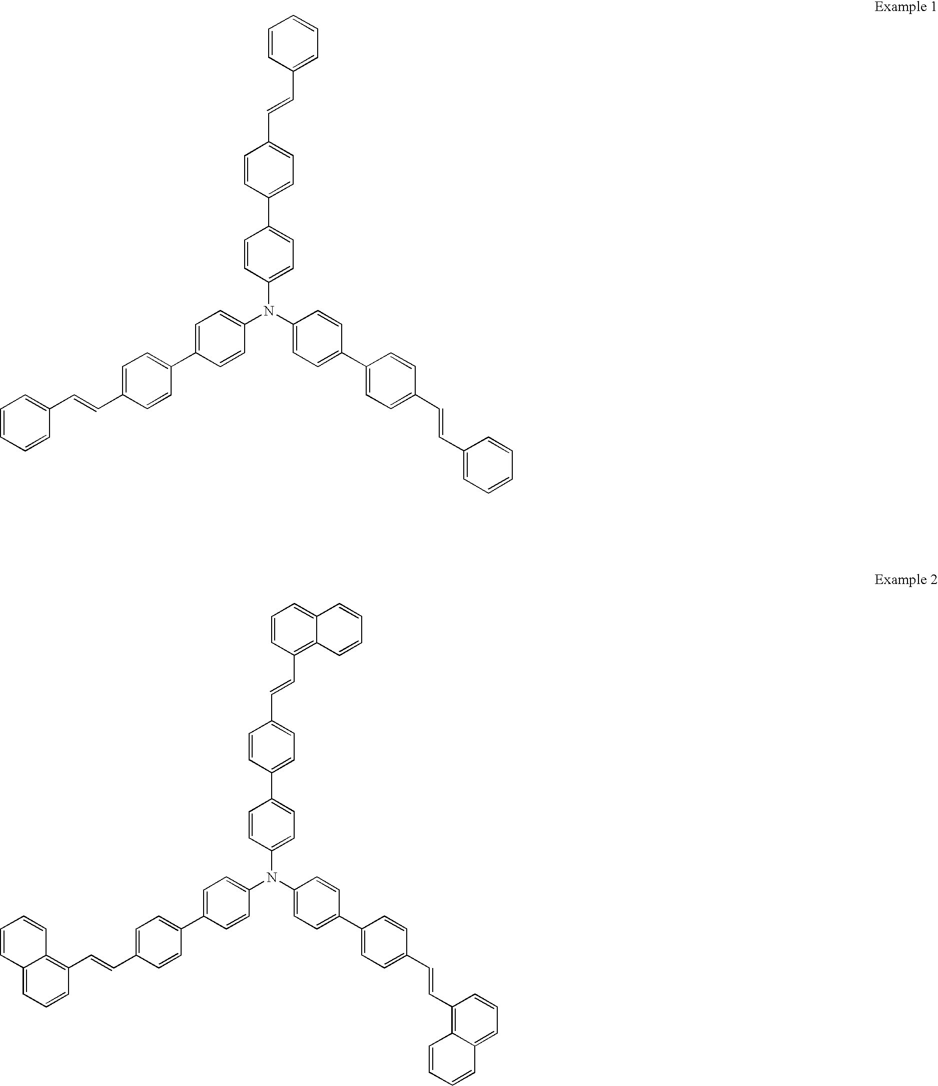

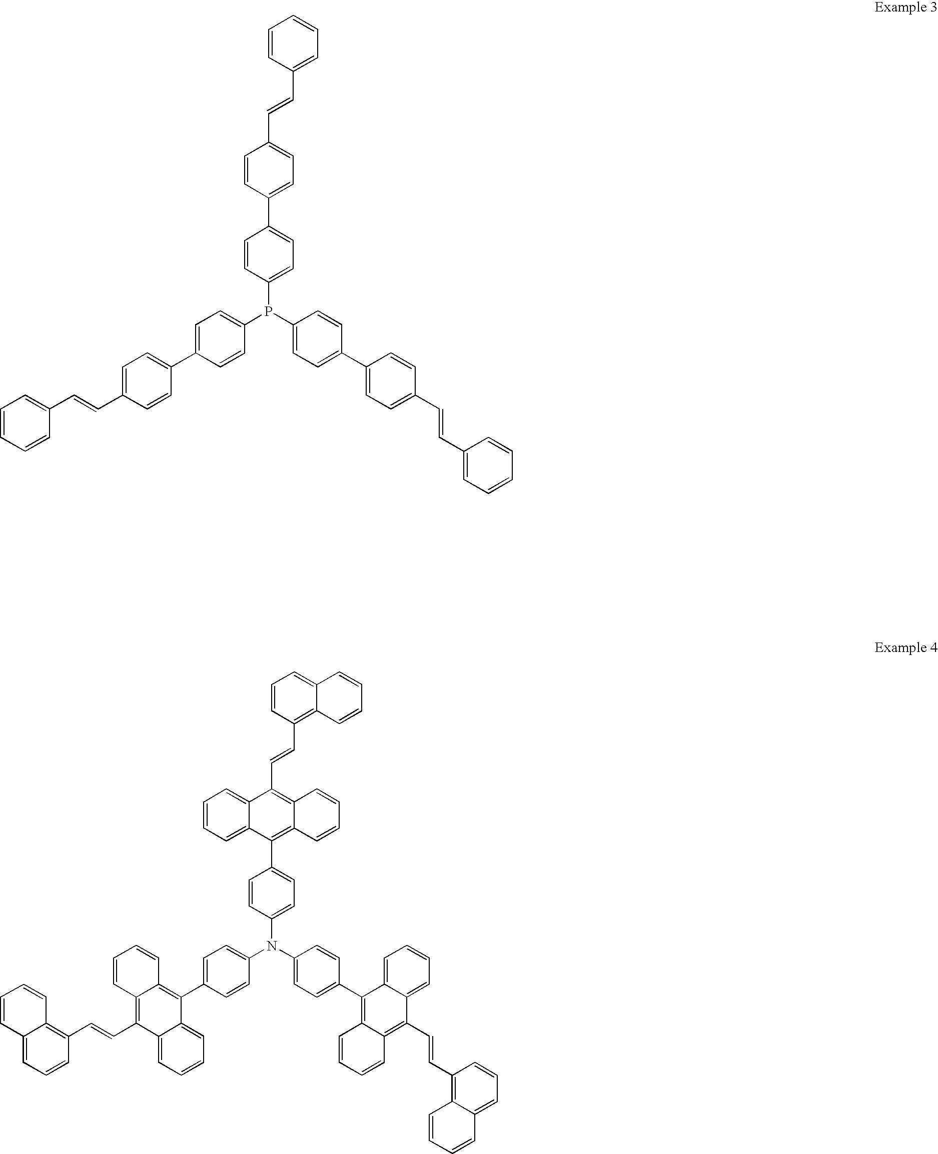

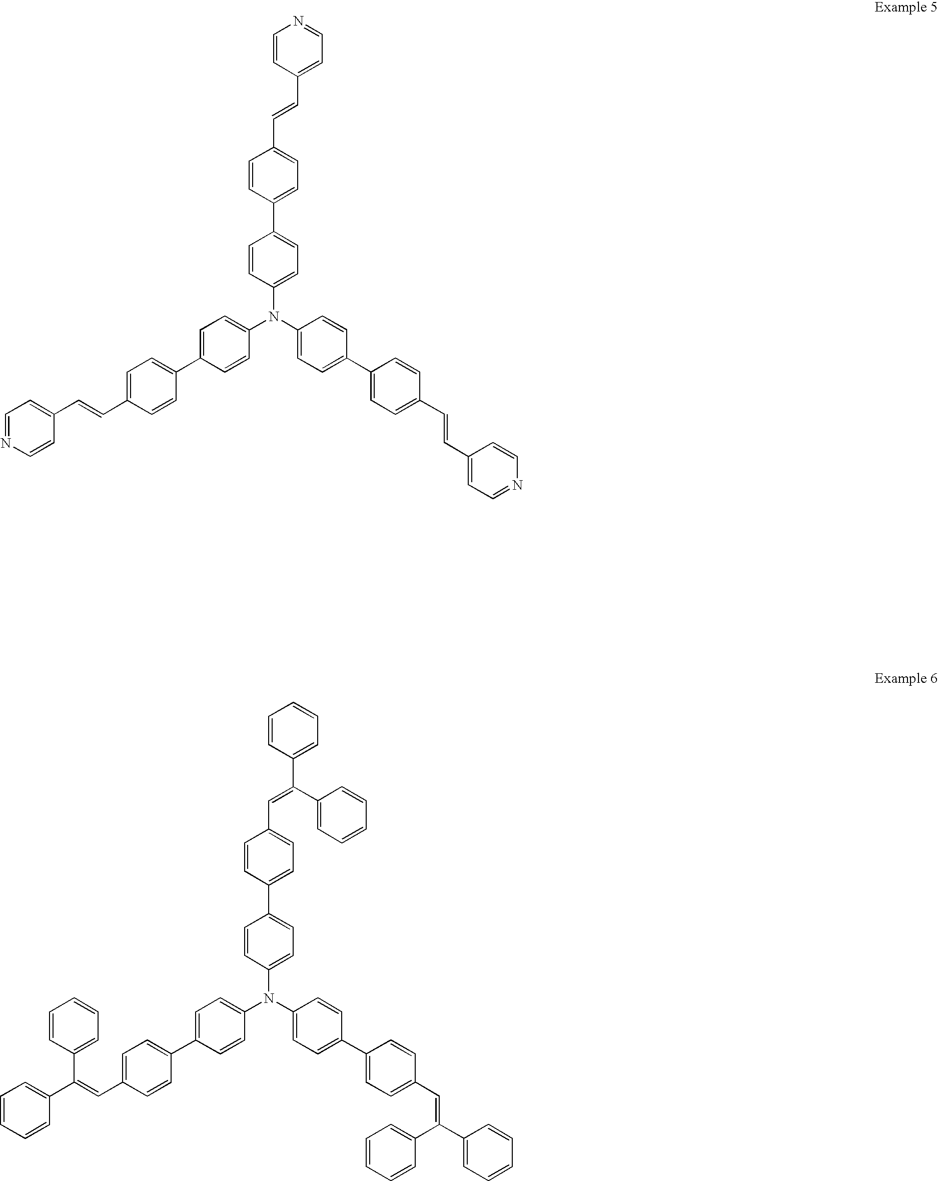

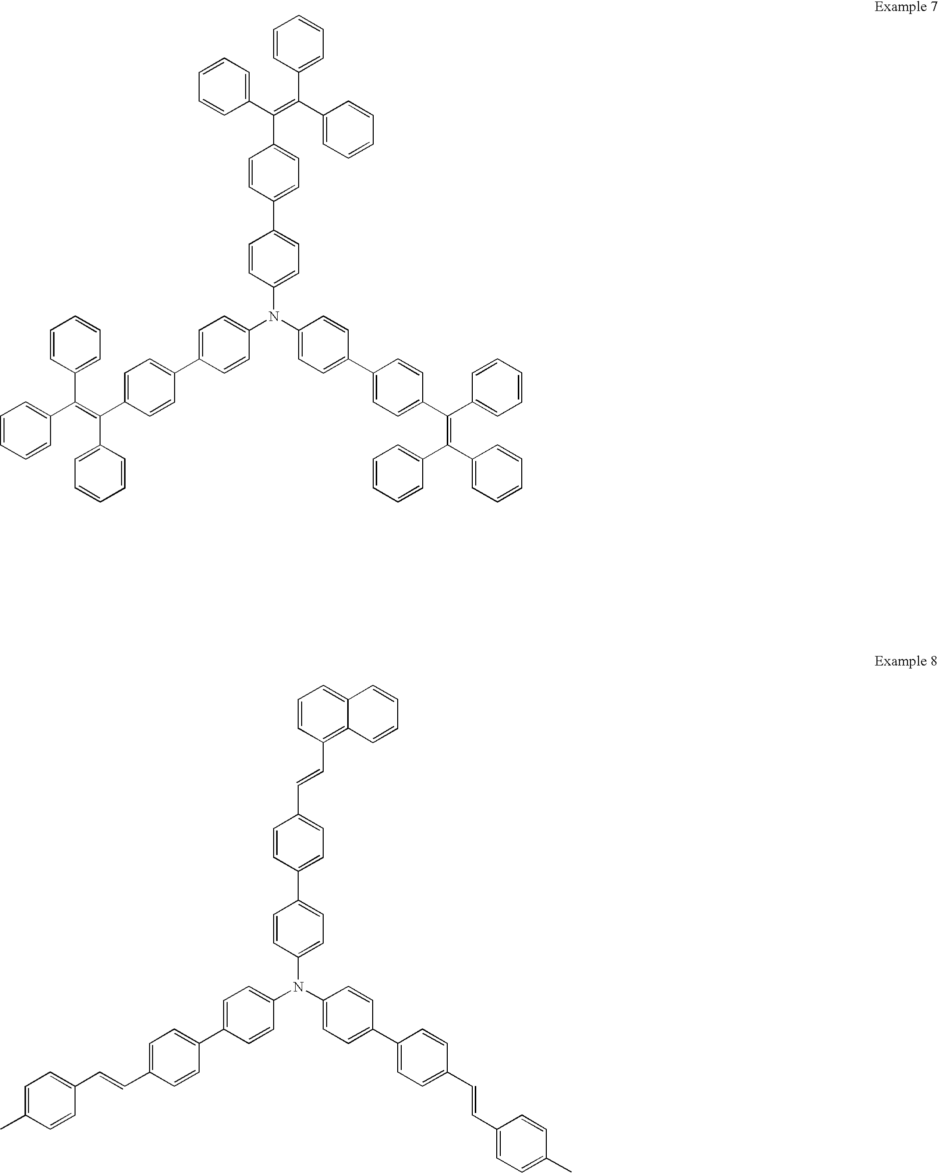

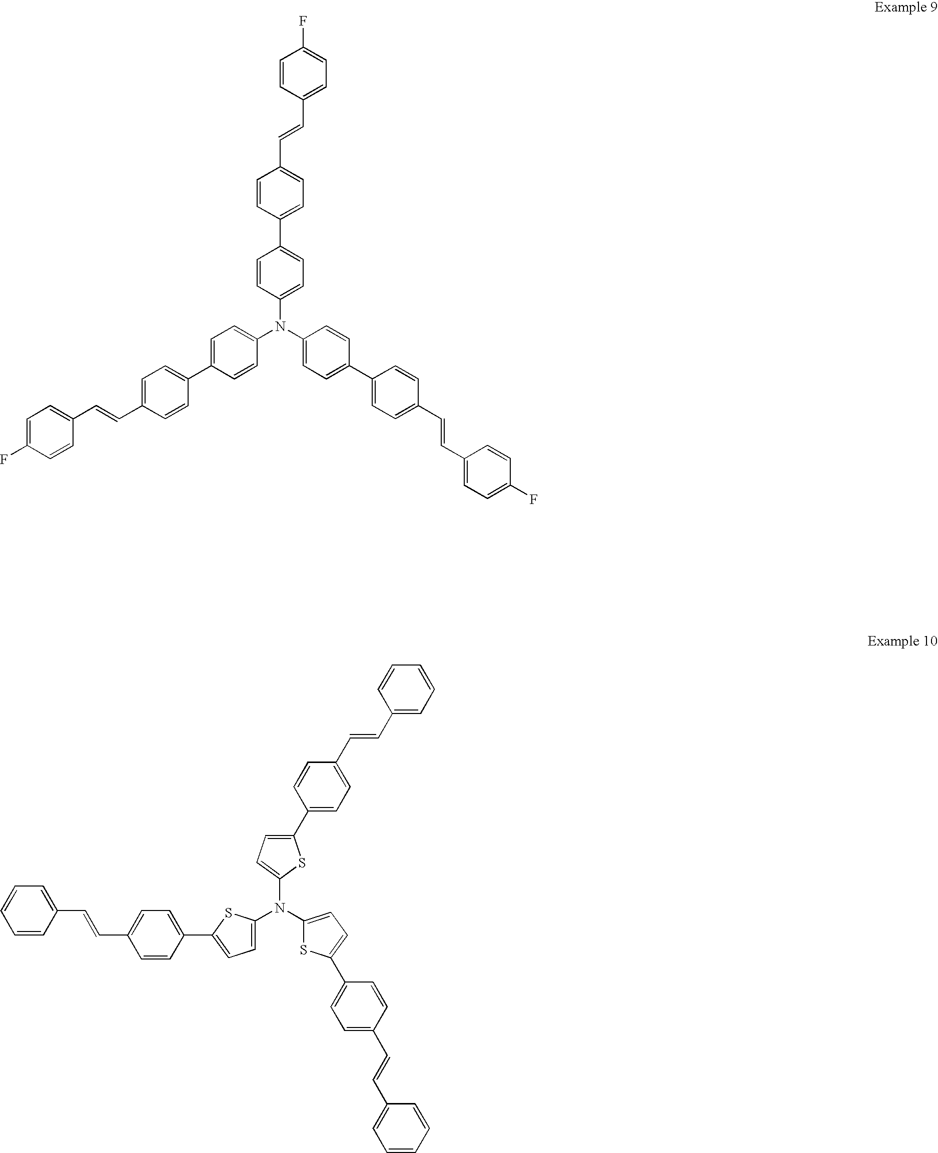

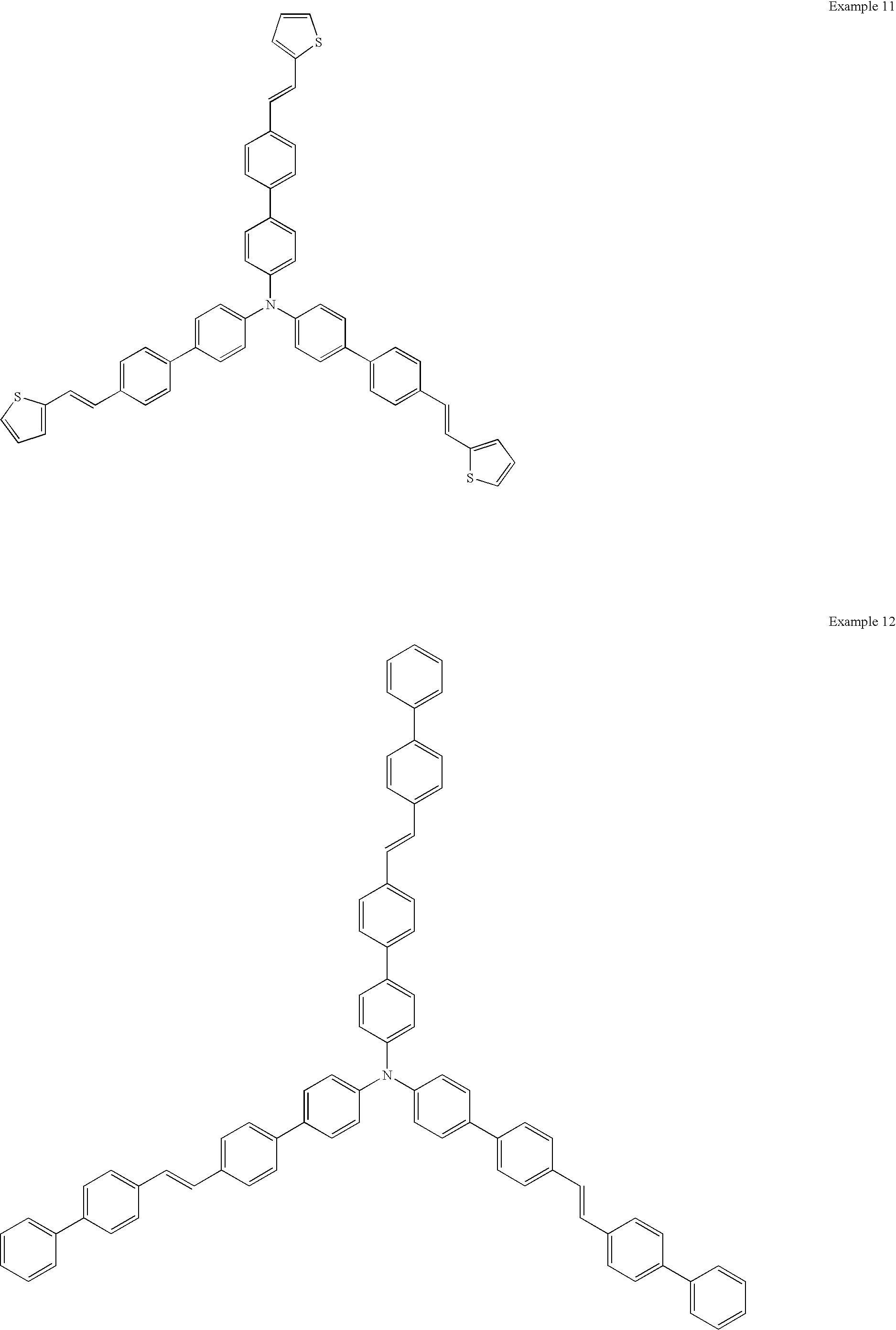

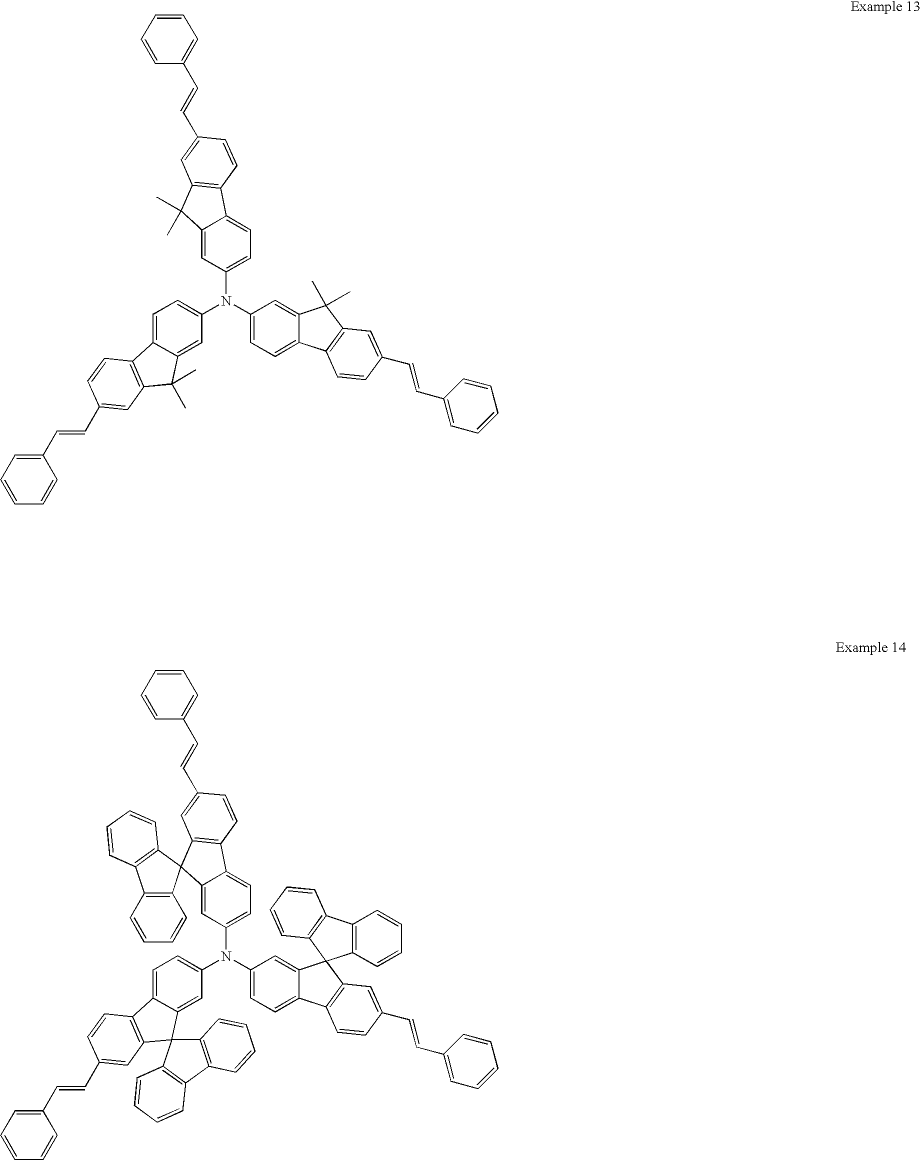

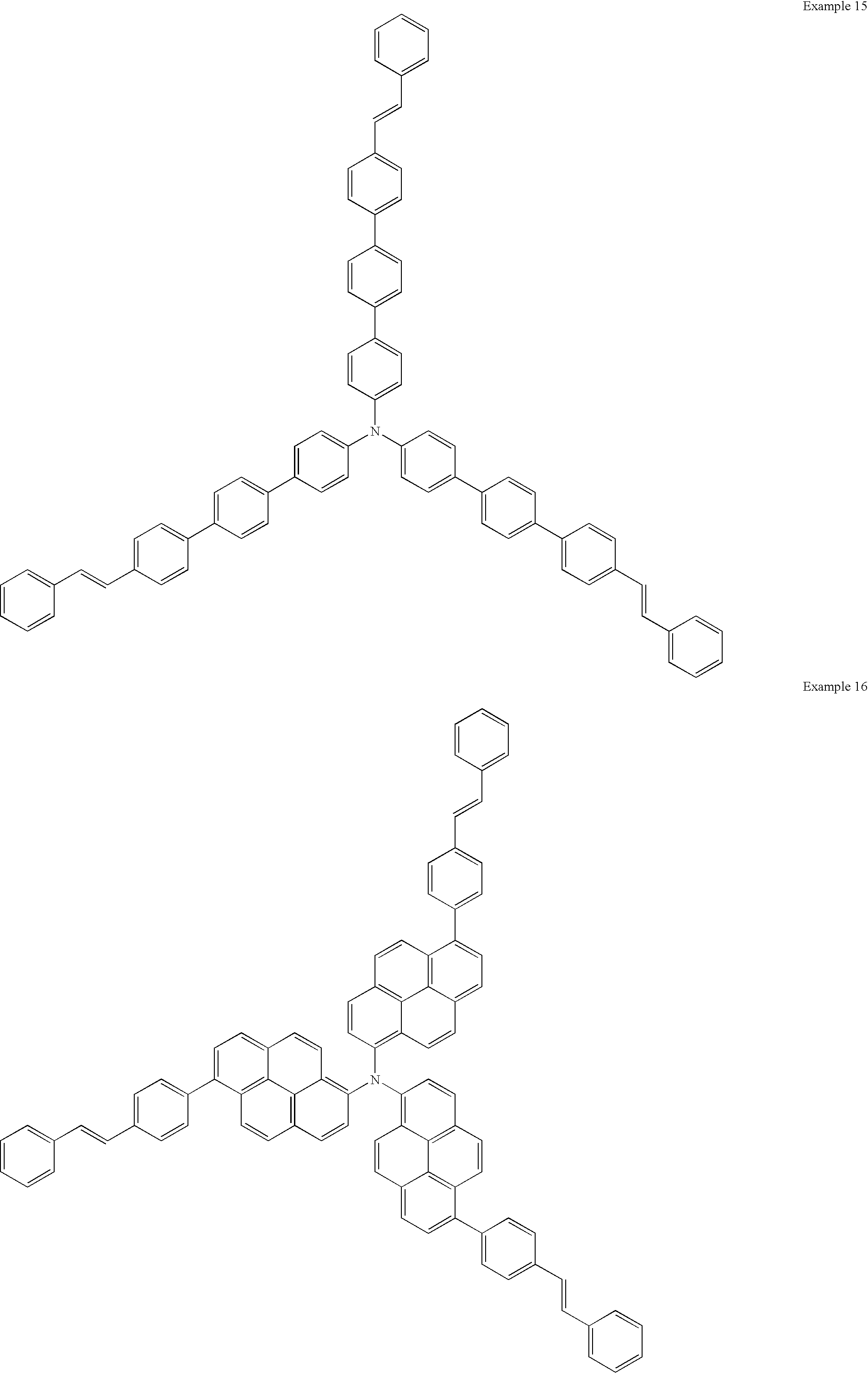

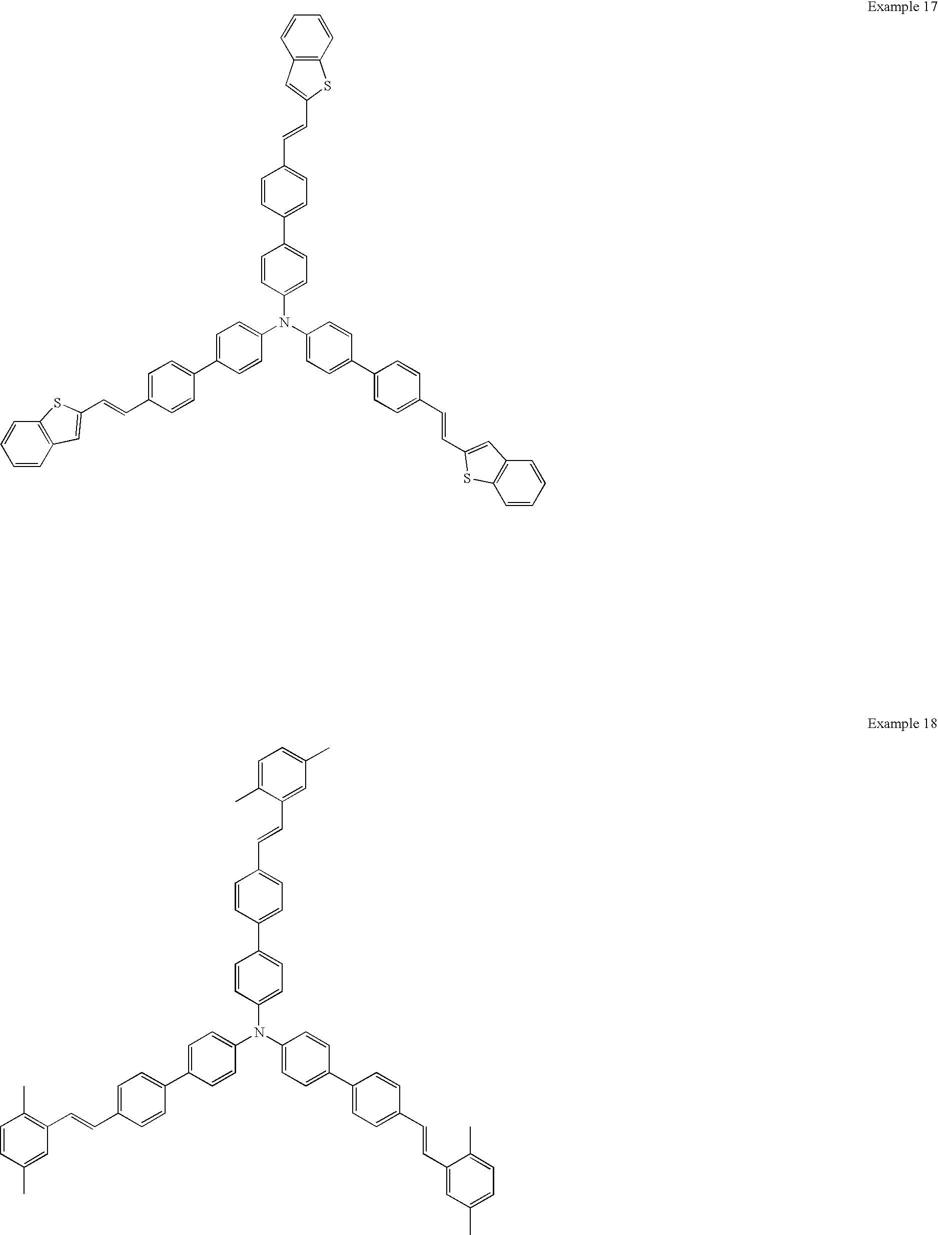

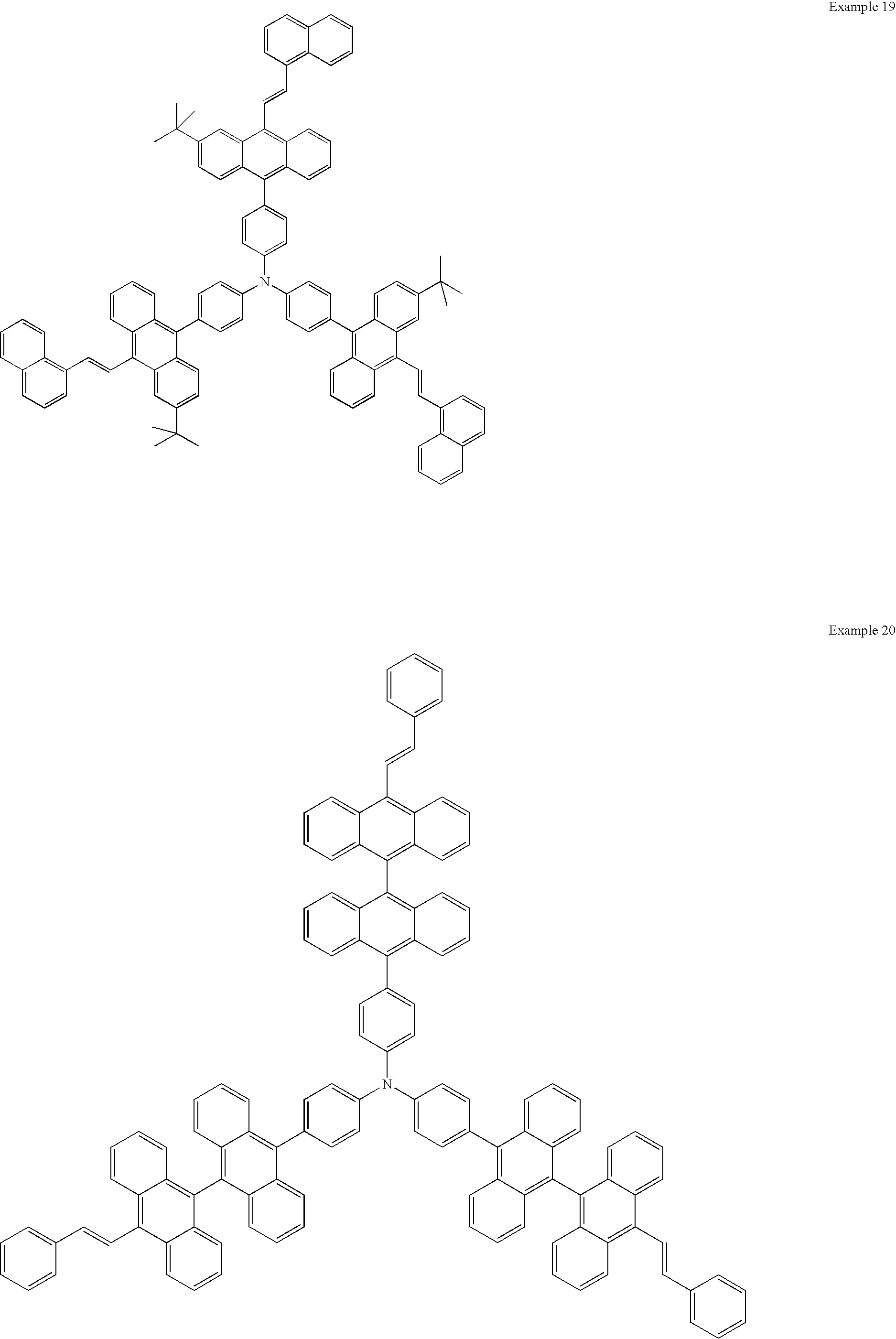

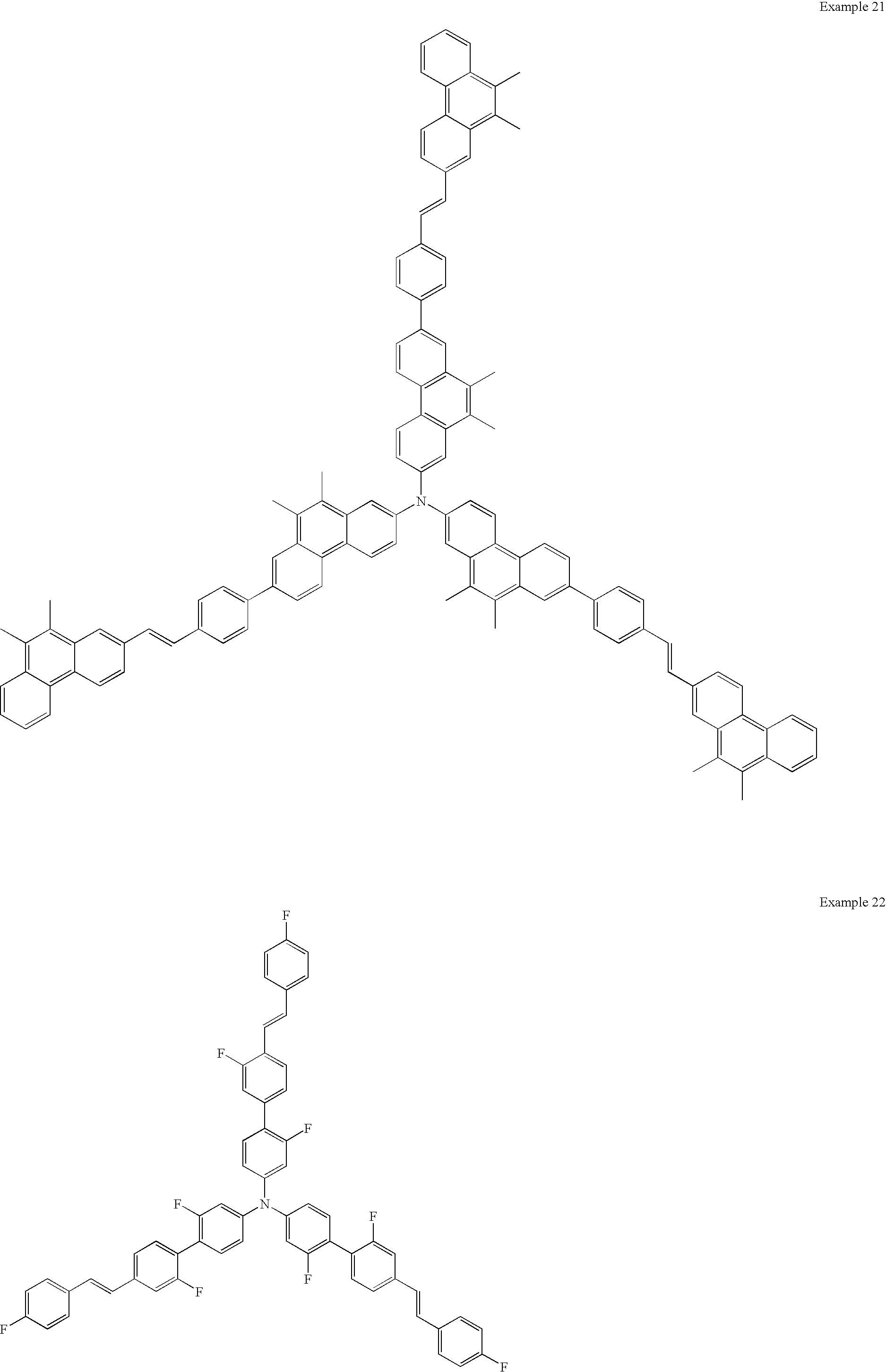

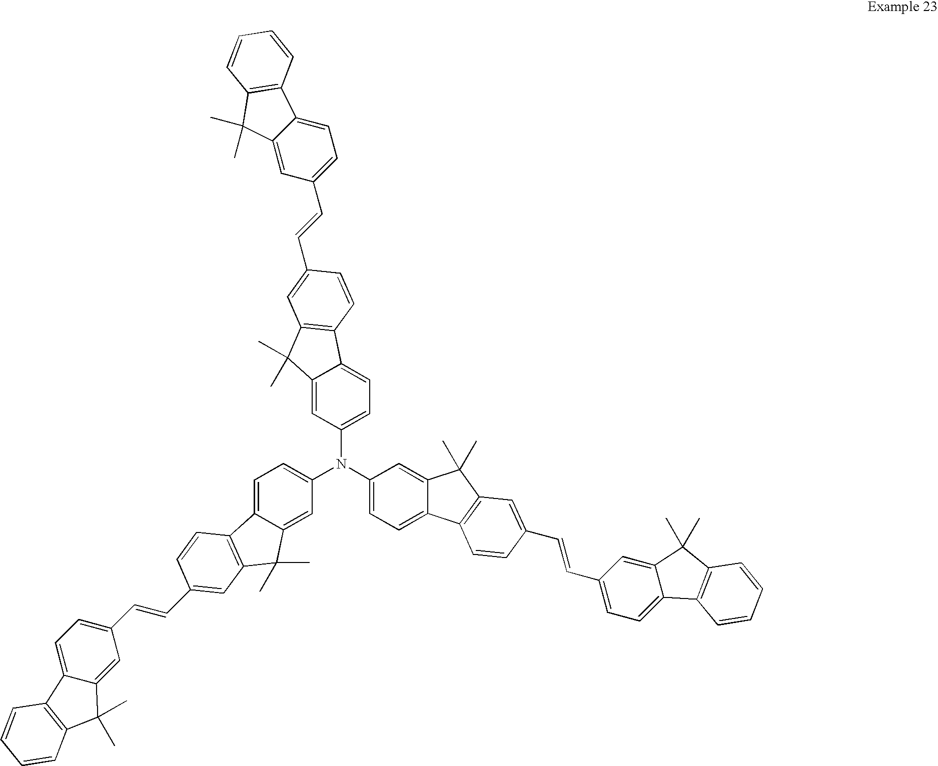

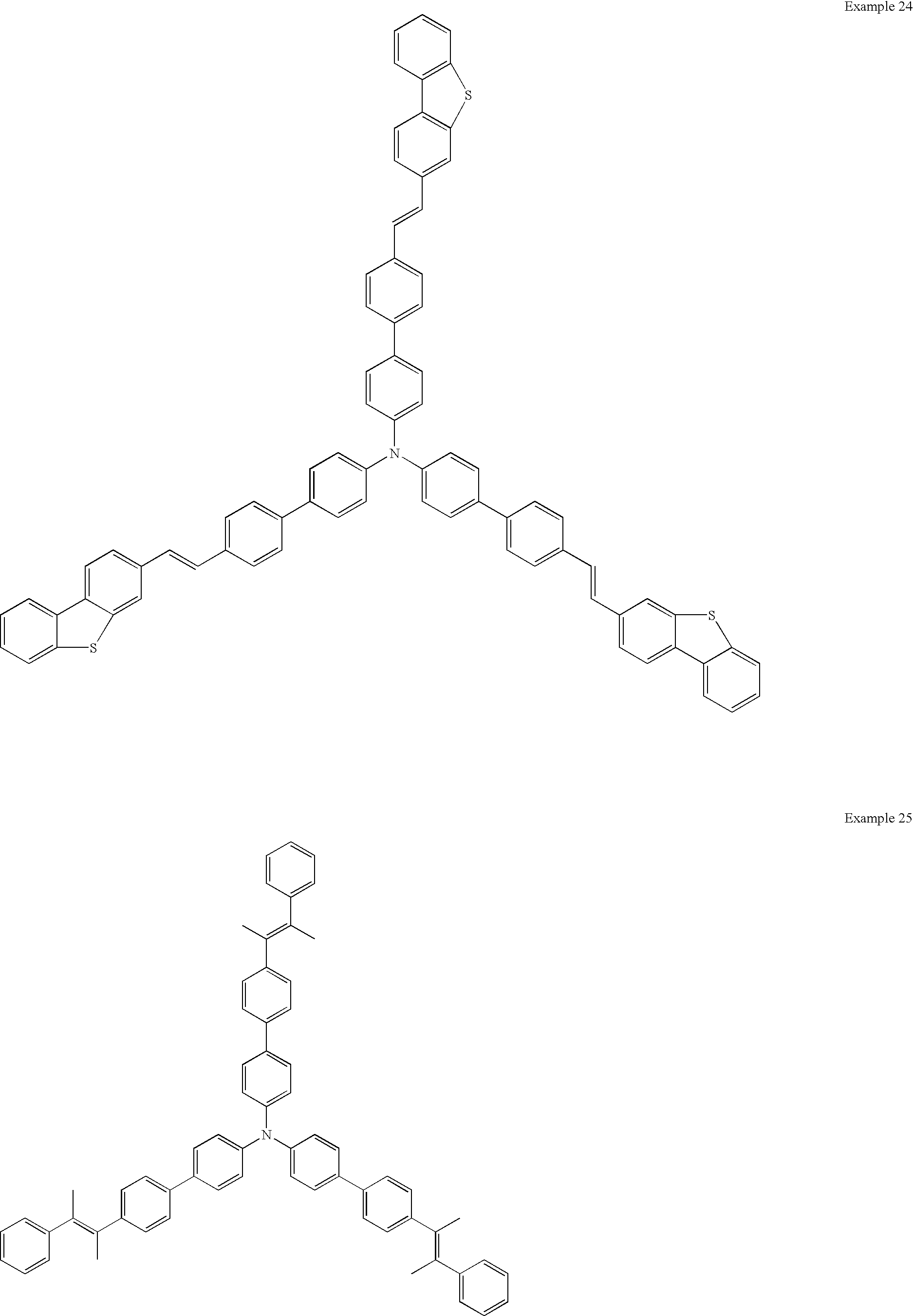

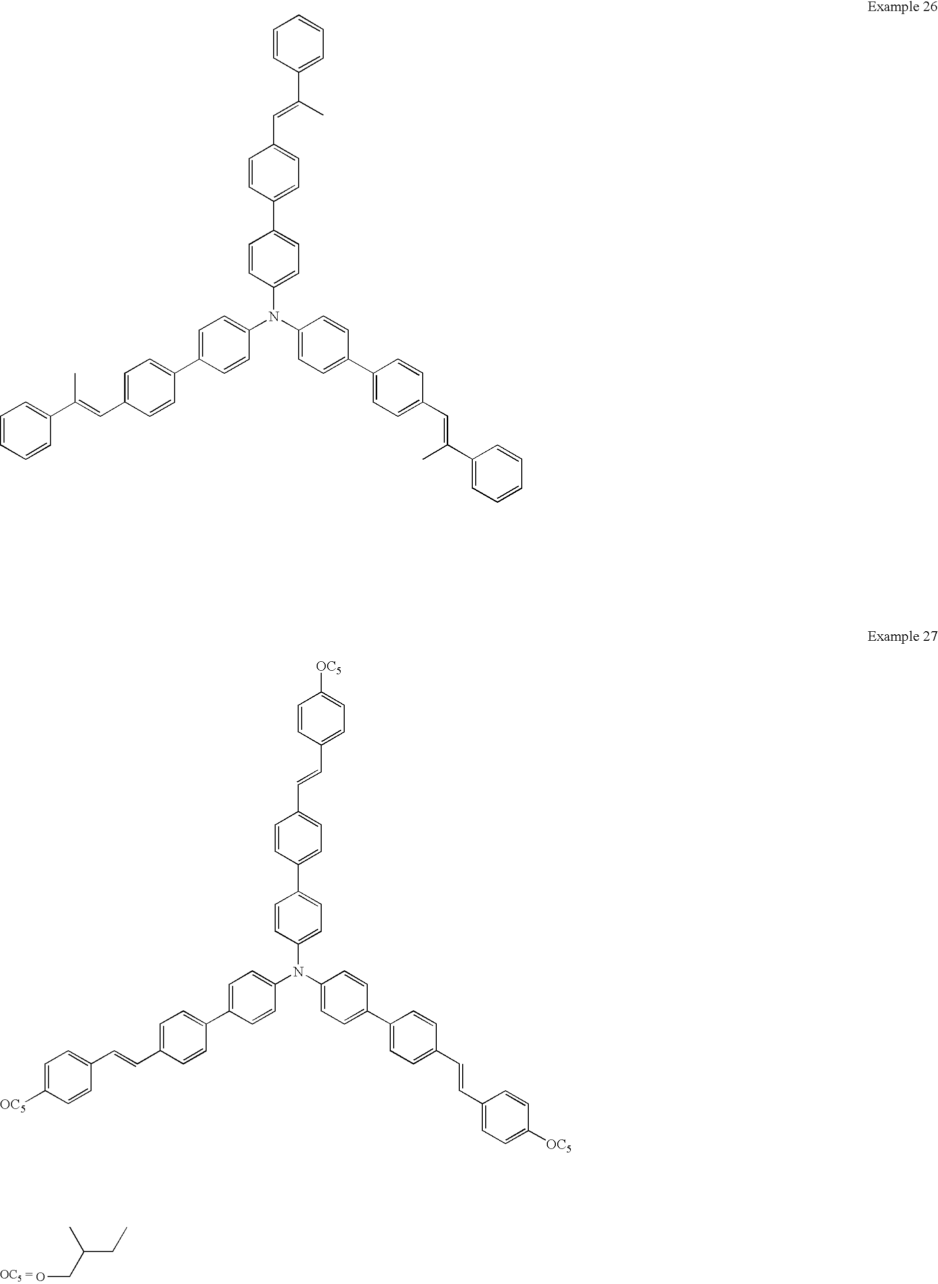

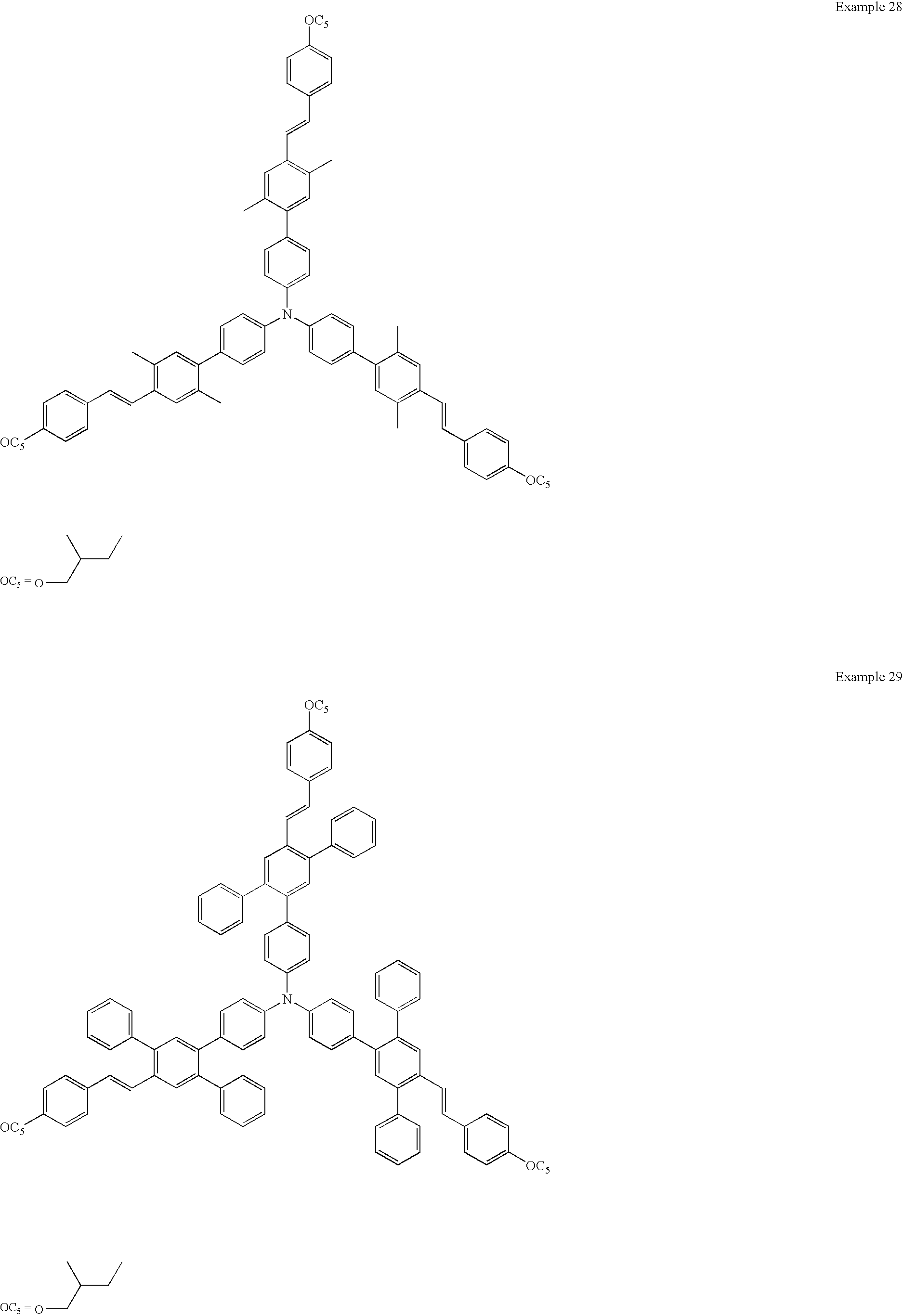

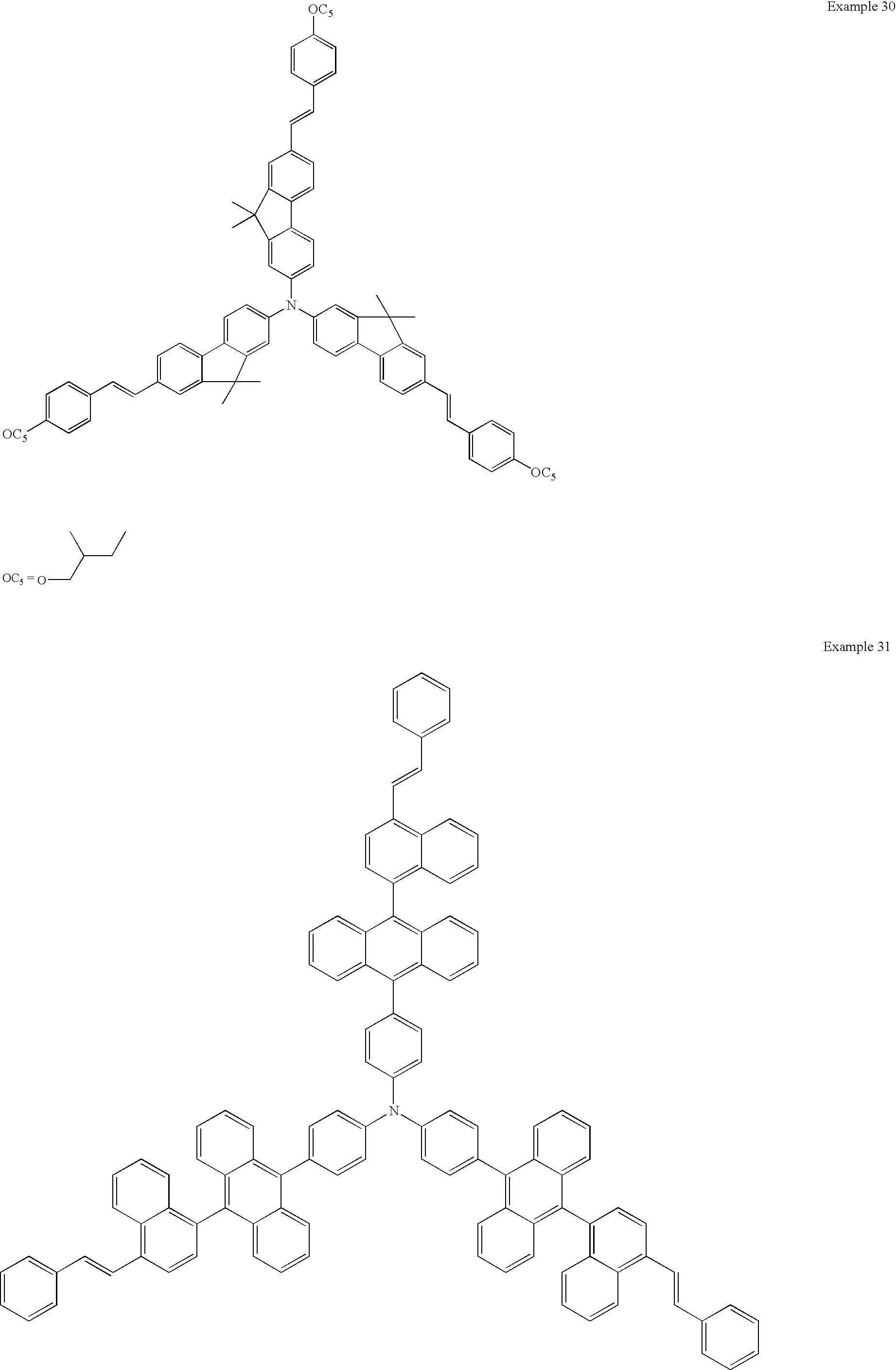

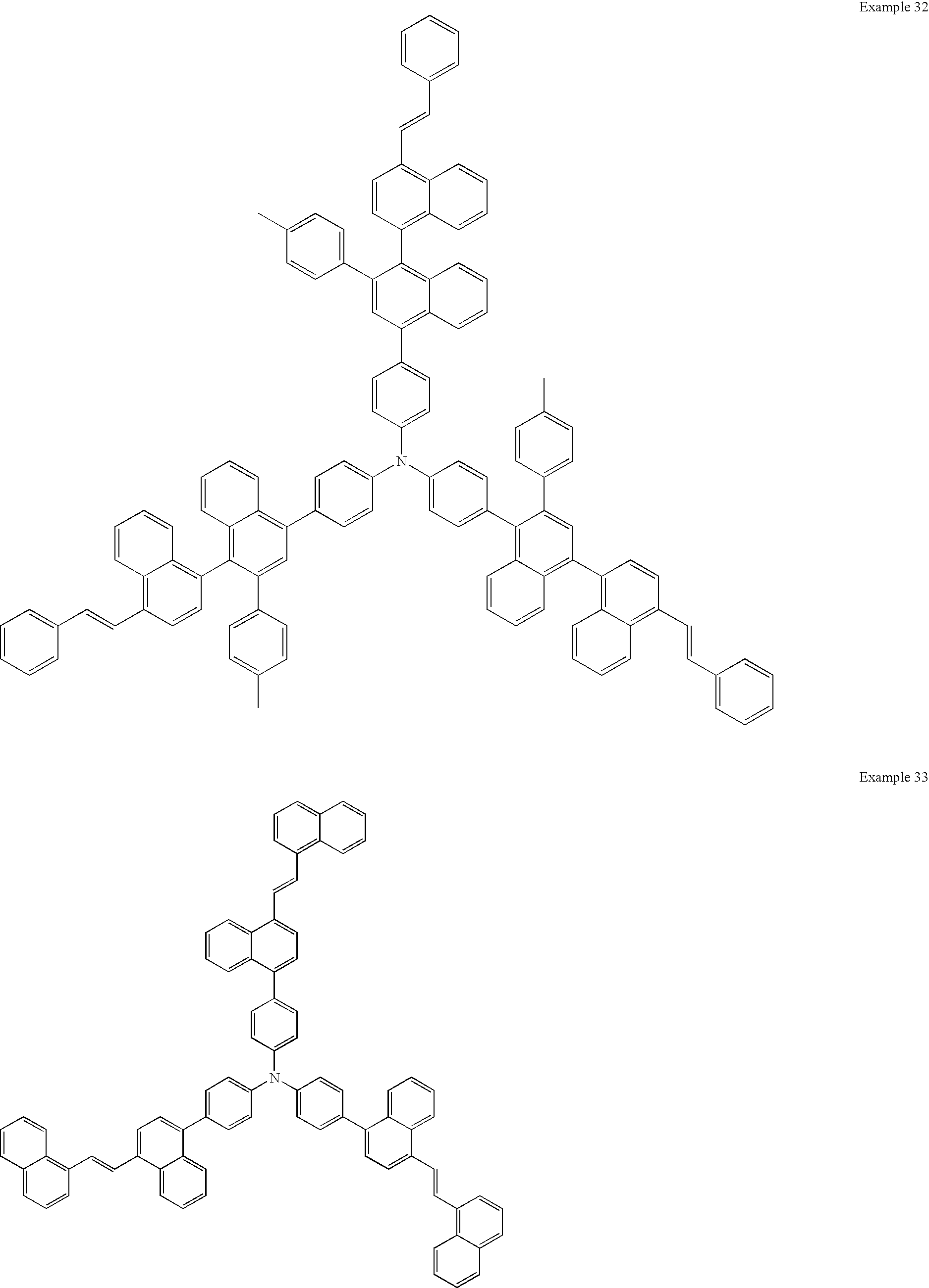

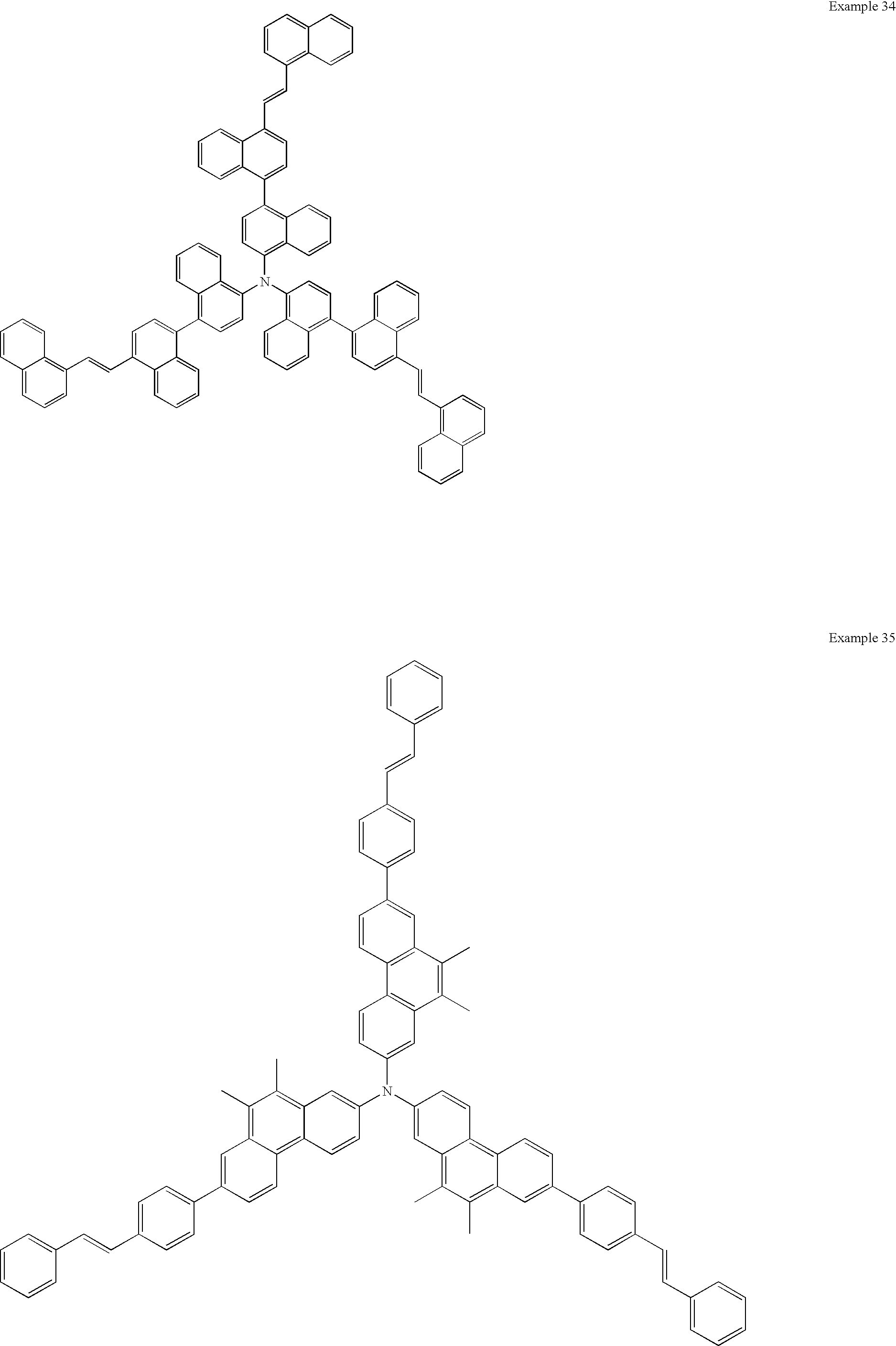

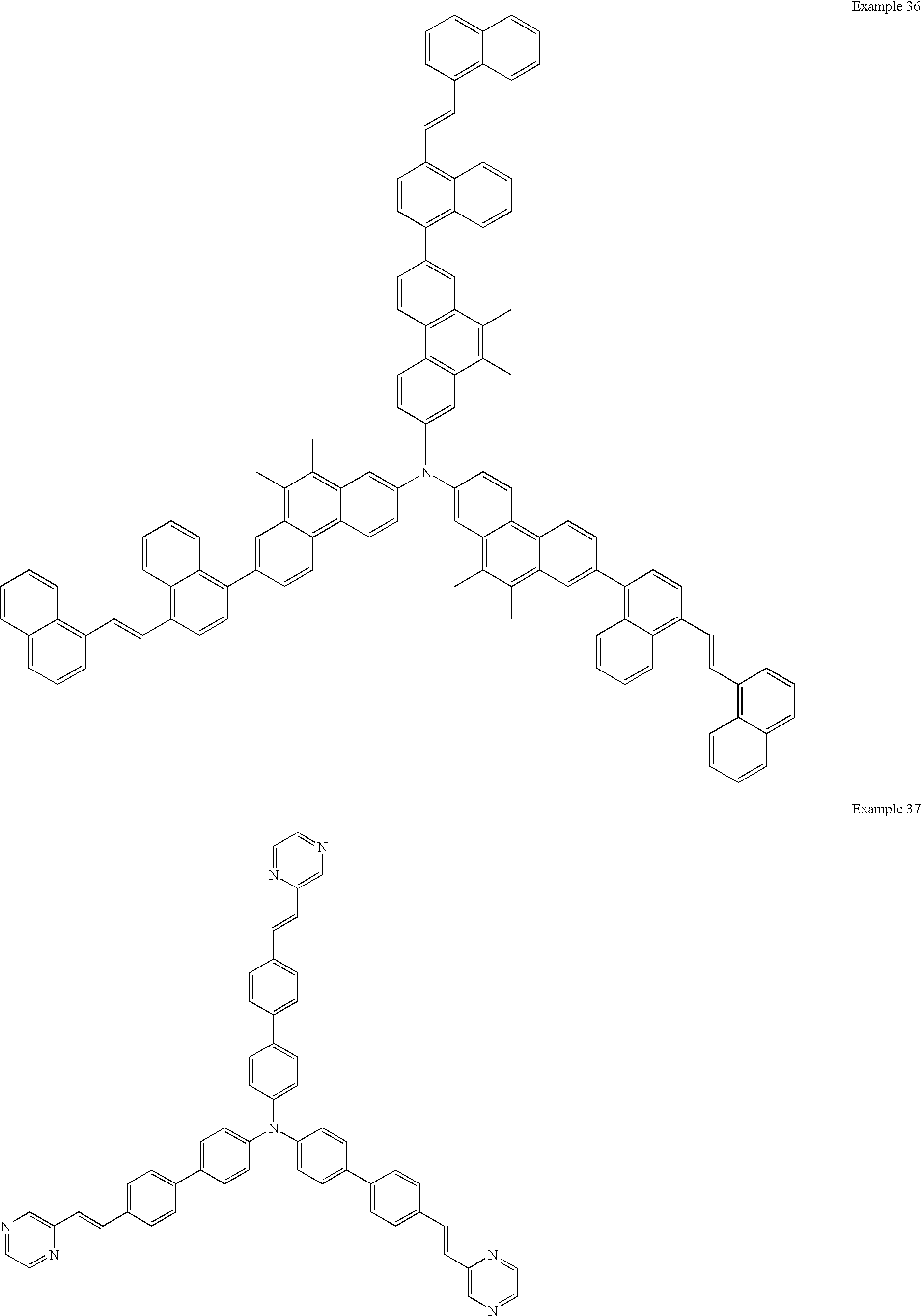

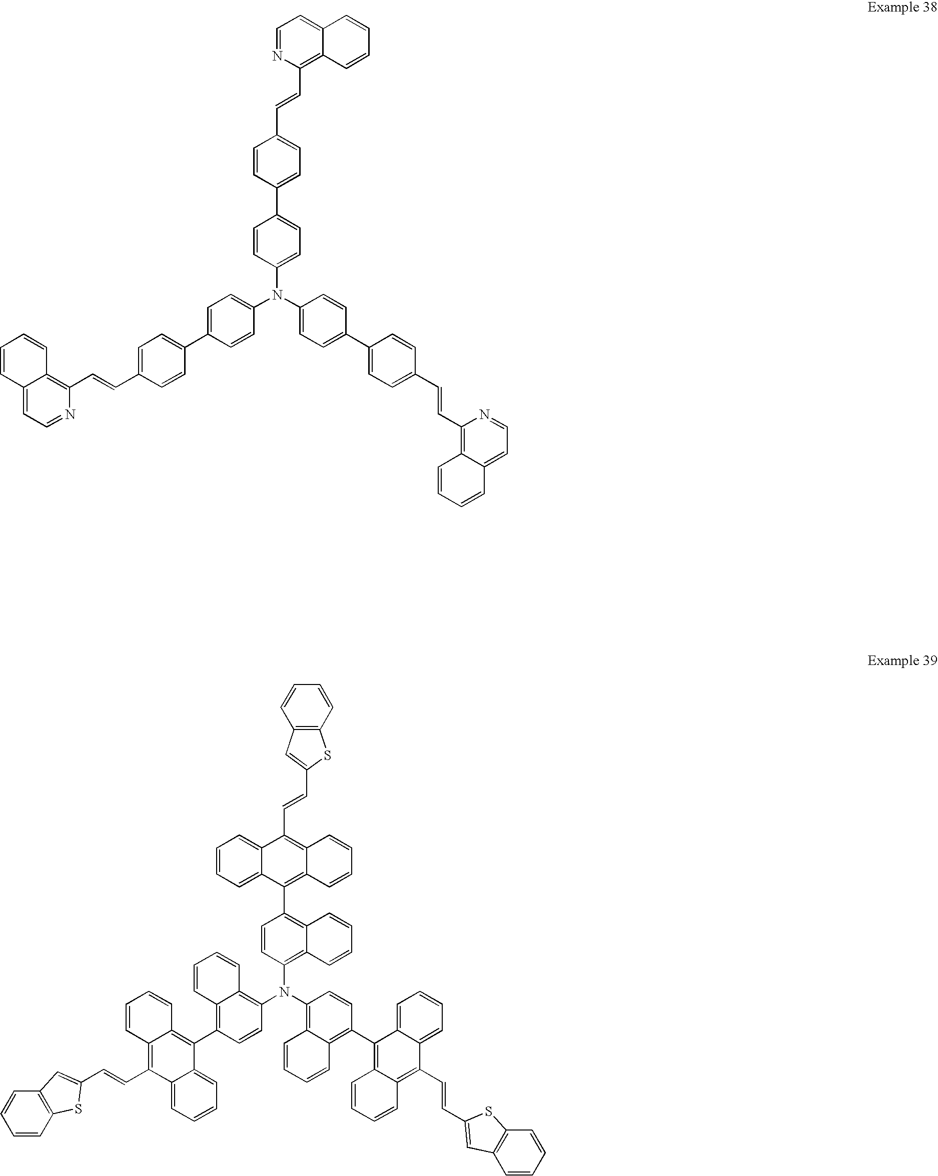

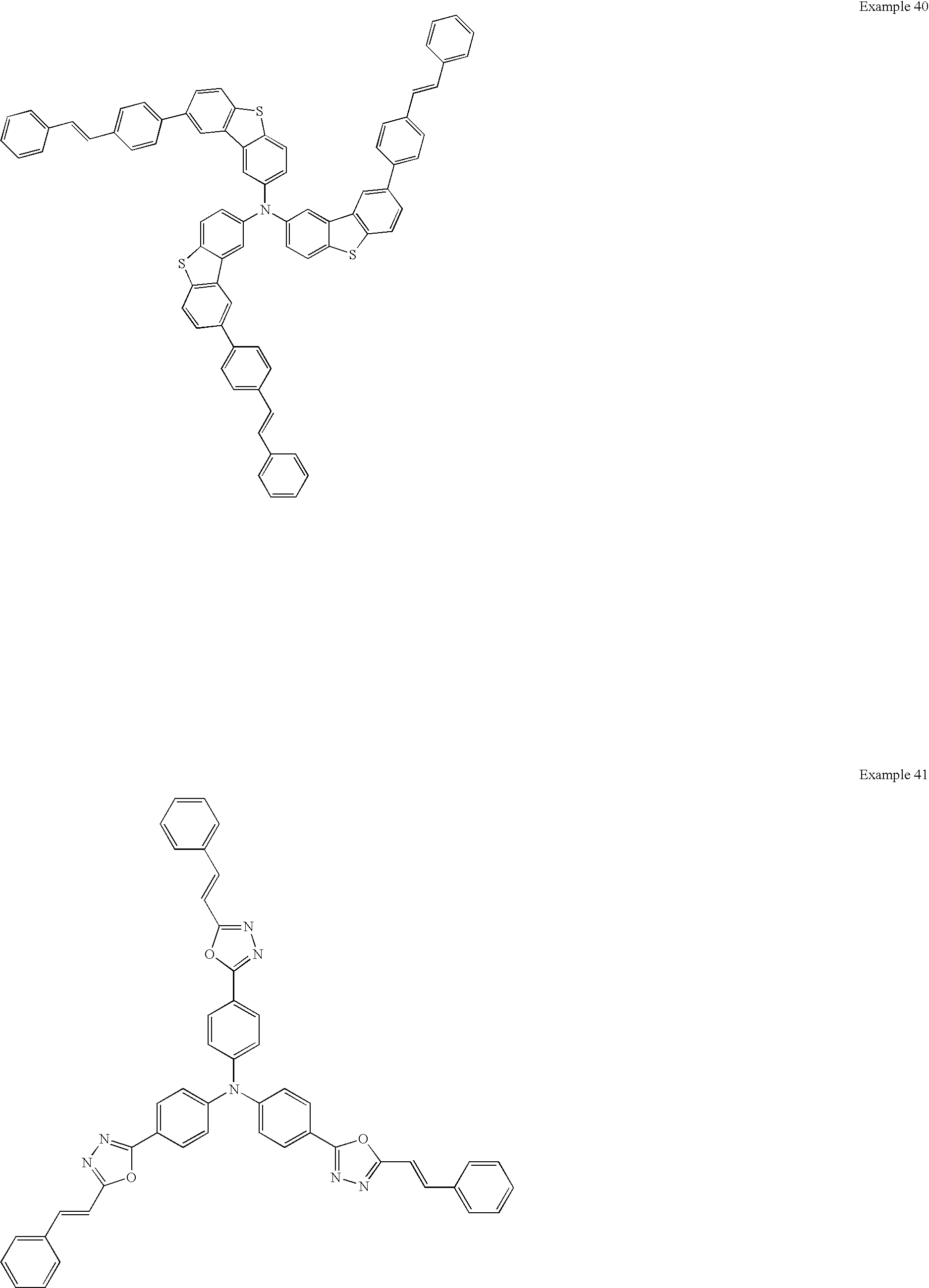

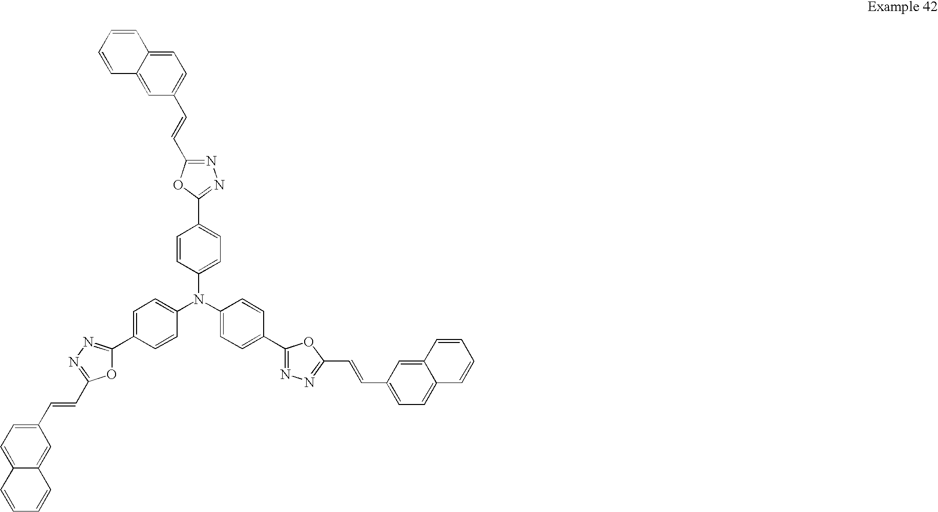

33. An organic electronic device comprising at least one compound according to claim 21.

34. An organic electronic device according to claim 33, wherein said device is selected from the group consisting of organic electroluminescent devices, organic field-effect transistors, organic thin-film transistors, organic light-emitting transistors, organic integrated circuits, organic solar cells, organic field-quench devices, and organic laser diodes.

35. An organic electroluminescent device comprising an anode, a cathode and at least one emitting layer, wherein said at least one emitting layer comprises at least one compound according to claim 21.

36. An organic electroluminescent device according to claim 35, wherein said at least one emitting layer comprises a mixture of said at least one compound and at least one host material.

37. An organic electroluminescent device according to claim 36, wherein said at least one host material is selected from the group consisting of oligoarylenes, oligoarylenes comprising condensed aromatic groups, oligoarylenevinylenes, polypodal metal complexes, hole-conducting compounds, electron-conducting compounds, ketones, phosphine oxides, sulfoxides, and atropisomers.

38. An organic electroluminescent device according to claim 36, wherein the proportion of said at least one compound in said mixture is between 0.1 and 99.0% by weight and the proportion of said at least one host material in said mixture is correspondingly between 1.0 and 99.9% by weight.

39. An organic electroluminescent device according to claim 35, further comprising at least one layer selected from the group consisting of hole-injection layers, hole-transport layers, electron-transport layers, electron-injection layers, and combinations thereof.

40. An organic electroluminescent device according to claim 35, wherein said at least one emitting layer comprises a plurality of emitting compounds, wherein said plurality of emitting compounds is present in the same layer or in different layers and wherein at least one of said emitting compounds has a structure of formula (1) 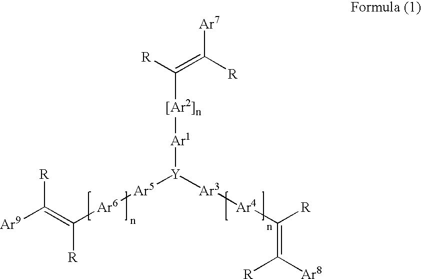
wherein
Y is on each occurrence N, P, As, Sb, P═O, As═O or Sb═O;
Ar¹, Ar², Ar³, Ar⁴, Ar⁵, and Ar⁶ are on each occurrence, identically or differently, a divalent aryl or heteroaryl group having up to 24 C atoms, which is optionally substituted by one or more radicals R¹;
Ar⁷, Ar⁸, and Ar⁹ are on each occurrence, identically or differently, a monovalent aryl or heteroaryl group having up to 24 C atoms, which is optionally substituted by one or more radicals R¹;
R is on each occurrence, identically or differently, H, CN, or a straight-chain, branched or cyclic alkyl group having up to 40 C atoms, which is optionally substituted by one or more radicals R², and wherein one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²—, and wherein one or more H atoms is optionally replaced by F, Cl, Br, I, CN, NO₂, or a monovalent aryl or heteroaryl group having up to 24 C atoms, which is optionally substituted by one or more radicals R¹;
R¹ is on each occurrence, identically or differently, H, F, Cl, Br, I, CN, NO₂, or a straight-chain, branched or cyclic alkyl, alkoxy or thioalkoxy group having up to 40 C atoms, which is optionally substituted by one or more radicals R², wherein one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²—, and wherein one or more H atoms is optionally replaced by F, Cl, Br, I, CN, NO₂, an aryl or heteroaryl group having up to 24 C atoms, which is optionally substituted by one or more radicals R², an aryloxy or heteroaryloxy group having up to 24 aromatic C atoms, which is optionally substituted by one or more radicals R², or a combination of two, three, four or five of these systems; and wherein two or more substituents R¹, on the same ring and/or on different rings, optionally define a monocyclic or polycyclic aliphatic or a monocyclic or polycyclic aromatic ring system with one another;
R² is on each occurrence, identically or differently, H or an aliphatic or aromatic hydrocarbon radical having up to 20 C atoms;
n is on each occurrence, identically or differently, 1, 2, 3, 4 or 5.
